(19) United States
(12) Patent Application Publication
(10) Pub. No.: US 2025/0127514 A1
(43) Pub. Date: Apr. 24, 2025

(54) SYSTEMS AND METHODS FOR TREATING HEART FAILURE BY REDIRECTING BLOOD FLOW IN THE AZYGOS VEIN

(71) Applicant: inQB8 Medical Technologies, LLC, Winchester, MA (US)

(72) Inventors: Arshad Quadri, West Hartford, CT (US); Jeremy Brent Ratz, Winchester, MA (US); Garrett Dallas Johnson, Costa Mesa, CA (US); Alec Rebh, Boylston, MA (US); Matthew Steven Lapointe, Framingham, MA (US); Brianna Parbattie Raghunath, Worcester, MA (US); Casey Nicole Martin, Worcester, MA (US); Audrey Bell, Wellesley, MA (US)

(21) Appl. No.: 18/919,289

(22) Filed: Oct. 17, 2024

Related U.S. Application Data
(60) Provisional application No. 63/591,382, filed on Oct. 18, 2023.
(60) Provisional application No. 63/665,885, filed on Jun. 28, 2024.

(51) Int. Cl. A61B 17/12; A61B 17/00

(52) U.S. Cl. CPC A61B 17/12036; A61B 17/12118; A61B 2017/00022

(57) ABSTRACT

A system and method for treating heart failure of a patient by occluding blood flow from the azygos vein to the superior vena cava while allowing blood to flow through a shunt from the right pulmonary artery to the azygos vein. The system can include an occluder with an expandable cage positioned in the azygos vein to selectively control the flow of blood to the superior vena cava. The occluder can expand and collapse based on pressure measurements received from implanted pressure sensors.

### INCORPORATION BY REFERENCE TO ANY PRIORITY APPLICATIONS

This application claims the benefit of priority under 35 U.S.C. § 119 (e) to U.S. Provisional Patent Application No. 63/591,382, filed Oct. 18, 2023, and U.S. Provisional Patent Application No. 63/665,885, filed Jun. 28, 2024. Both of these applications are hereby incorporated by reference herein in their entireties. Any and all applications for which a foreign or domestic priority claim is identified in the Application Data Sheet as filed with the present application are hereby incorporated by reference under 37 CFR 1.57.

### BACKGROUND

### Field of the Technology

Embodiments of this application are directed to systems, methods and devices for treating heart failure by redirecting blood flow in the azygos vein.

### Description of the Related Art

Heart failure, or congestive heart failure, can occur when the heart fails to efficiently pump blood. Certain cardiovascular conditions, including narrowed arteries in the heart and high blood pressure, can gradually weaken and/or stiffen the heart muscle, reducing cardiac efficiency. As cardiac output decreases, blood pressure drops and blood circulation slows. Fluid can build up in the lungs, causing shortness of breath. Reduced blood flow and increased fluid further compromise the heart, and can eventually become life-threatening.

At the same time, the kidneys experience a drop in renal blood pressure as the heart begins to fail. In response, renal sympathetic activation releases hormones, including renin, angiotensin, and aldosterone to increase water and sodium retention as well as increase extracellular fluid in an attempt to raise the blood pressure and drive blood back to the heart. The increased pressure and fluid exacerbate the stress on the compromised heart, further accelerating the failure mechanism.

Heart failure can be initially treated by lifestyle changes to reduce the load on the heart, but in many cases, medication and/or renal intervention (such as ablation) may be used to reduce blood pressure and fluid buildup. As cases progress in severity, surgical intervention, such as coronary bypass, stent placement, heart valve repair or replacement, implantation of cardioverter-defibrillator, use of ventricular assist device, or heart transplant, may become necessary.

### SUMMARY

In patients with heart failure, the heart may not be able to keep up with the effective circulating volume. It is believed that diverting blood from the arterial system to the venous system, for example using a shunt, may be help reduce a workload of the patient's heart. Since veins are more compliant than arteries, it is believed that the venous system has sufficient capacitance to accommodate additional blood from the arterial system and reduce arterial pressure. In particular, when blood is shunted from the pulmonary artery into the azygos vein, it is believed veins like the accessory hemiazygos vein or the hemiazygos vein may dilate and effectively create a tank of extra blood. Diverting blood away from the pulmonary artery may reduce pulmonary artery pressure to relieve pulmonary hypertension and consequently reduce afterload of a right ventricle of the patient.

Shunting blood from the pulmonary artery into the azygos vein, alone, may be insufficient to increase overall capacitance in the venous system because the additional blood from the right pulmonary artery may flow directly into the superior vena cava and into the right atrium. At least partially or fully occluding blood flow from the azygos vein to the superior vena cava for a period of time, may allow at least some of the diverted blood to flow through and accumulate in other blood vessels (e.g., the accessory hemiazygos vein, the hemiazygos vein, or intercostal veins), thus increasing venous capacitance and reducing arterial pressure, and thereby reducing preload of a right ventricle of the patient. Moreover, at least partially or fully occluding blood flow from the azygos vein to the superior vein, may increase the amount of blood flowing in the azygos vein in a retrograde direction. When the occlusion to the superior vena cava is removed, blood may continue to flow antegrade from the hemiazygos or accessory hemiazygos veins toward the azygos vein and into the superior vena cava.

Certain aspects of the present application are directed to methods, systems and devices for treating heart failure. Certain aspects of the present application are directed to methods, systems and devices that divert or redirect blood flow in the azygos vein.

A system for treating heart failure of a patient can include a shunt configured to direct blood flow from an artery to a vein, for example a pulmonary artery to an azygos vein. The shunt can reduce pressure in the artery. The system can include an expandable occluder configured to be positioned within the azygos vein to at least partially occlude blood flow through the azygos vein into a superior vena cava. The occluder can selectively cause blood to flow retrograde in the azygos vein, forming a thoracic tank. The system can include an implantable control unit configured to regulate expansion of the expandable occluder to cause blood flow from the pulmonary artery through the shunt to the azygos vein in a retrograde direction through the azygos vein.

In some embodiments, the shunt can include a check valve. The check valve can selectively allow blood to pass through the shunt. In some embodiments, the shunt is configured to passively allow blood to flow from the pulmonary artery to the azygos vein. In some embodiments, the shunt can include a first flared end configured to seal against a puncture in a pulmonary artery and a second flared end configured to seal against a puncture in an azygos vein. The flared ends can secure the shunt in place. In some embodiments, the system can include an anchoring element configured to anchor the expandable occluder within the azygos vein. In some embodiments, the anchoring element can include an expandable stent configured to engage inner walls of the azygos vein. In some embodiments, the anchoring element is removably connectable to the expandable occluder.

In some embodiments, the system can include a drive motor connectable to the expandable occluder, the drive motor configured to expand and collapse the expandable occluder. In some embodiments, the drive motor is configured to be positioned in a superior vena cava. In some embodiments, the system can include a lead connectable to the drive motor and the implantable control unit. In some embodiments, the lead is configured to be positioned in a right subclavian vein. In some embodiments, the control unit is configured to be implanted subcutaneously.

In some embodiments, the system can include one or more implantable pressure sensors, the implantable control unit configured to receive pressure measurements from the one or more implantable pressure sensors. The implantable control unit can be configured to control the expandable occluder based on the pressure measurements. For example, the implantable control unit can cause the expandable occluder to expand and collapse when pressure measurements are above or below a threshold. In some embodiments, the one or more implantable pressure sensors are configured to be positioned in the superior vena cava. In some embodiments, the one or more implantable pressure sensors are positioned between in the azygos vein. In some embodiments, the implantable control unit can include an accelerometer and/or an electrocardiogram. The accelerometer can measure activity of a patient and the electrocardiogram can measure electrical activity of a heart of a patient. These measurements can contextualize the pressure readings and affect the implantable control unit's decision to expand or collapse the expandable occluder.

A method of treating heart failure of a patient can include positioning a shunt to direct blood flow from a pulmonary artery to an azygos vein. The shunt can be positioned using a guidewire. The method can include positioning an expandable occluder within the azygos vein to at least partially occlude blood flow through the azygos vein into a superior vena cava. The expandable occluder can be positioned using a guide sheath. The method can include expanding the expandable occluder based on feedback received from one or more pressure sensors to at least partially occlude blood flow through the azygos vein such that blood flows from the pulmonary artery through the shunt to the azygos vein in a retrograde direction through the azygos vein.

In some embodiments, receiving the feedback from the one or more pressure sensors includes measuring a first pressure measurement in the azygos vein using a first implantable sensor positioned in the azygos vein and a second pressure measurement in a superior vena cava. In some embodiments, the method can include determining a central venous pressure based on the feedback from the one or more pressure sensors, for example the pressure in the superior vena cava. In some embodiments, expanding the expandable occluder based on feedback received from the one or more pressure sensors includes expanding the expandable occluder when central venous pressure exceeds a threshold pressure value. In some embodiments, the threshold pressure value is 15 mmHg. In some embodiments, the method can include collapsing the expandable occluder when central venous pressure falls below a threshold pressure value. In some embodiments, the threshold pressure value is 5 mmHg. The expandable occluder can also expand and collapse based on azygos vein pressure, a pressure differential between the pressure measurements, or other pressure measurements.

In some embodiments, the method can include collecting, by an accelerometer, one or more activity measurements. In some embodiments, the method can expanding the expandable occluder based at least in part on the one or more activity measurements. In some embodiments, the method can include receiving, by an electrocardiogram, one or more electrical activity measurements. In some embodiments, the method can expanding the expandable occluder based at least in part on the one or more electrical activity measurements. In some embodiments, expanding the expandable occluder includes moving, by a drive motor, a first end of the expandable occluder closer to a second end of the expandable occluder and moving, by the drive motor, the first end of the expandable occluder away from the second end of the expandable occluder. Moving the first end and the second end closer can cause the expandable occluder to expand. Moving the first end and the second end away from each other can cause the expandable occluder to collapse. In some embodiments, the method can include transmitting the feedback received from the one or more pressure sensors to an external device.

A method of treating heart failure of a patient can include providing an expandable occluder comprising a first cap, a second cap, and a plurality of members. Each member can include a first end positioned on the first cap and a second end positioned on the second cap. The method can include rotating, with a drive line attached to the first cap, the first cap with respect to the second cap. Rotating the first cap with respect to the second cap in a first direction can cause the plurality of members to expand radially outward. Rotating the first cap with respect to the second cap in a second direction can cause the plurality of members to expand radially inward.

The expandable occluder can be positioned in an azygos vein. The method can include expanding the expandable occluder based on feedback received from one or more pressure sensors to at least partially occlude blood flow through the azygos vein. Receiving the feedback from the one or more pressure sensors can include measuring a first pressure measurement in the azygos vein using a first implantable sensor positioned in the azygos vein and a second pressure measurement in a superior vena cava. The method can include determining a central venous pressure based on the feedback from the one or more pressure sensors. Expanding the expandable occluder based on feedback received from the one or more pressure sensors can include expanding the expandable occluder when central venous pressure exceeds a threshold pressure value. The threshold pressure value can be 15 mmHg. The method can include collapsing the expandable occluder when central venous pressure falls below a threshold pressure value. The threshold pressure value can be 5 mmHg. The method can include collecting, by an accelerometer, one or more activity measurements, and

expanding the expandable occluder based at least in part on the one or more activity measurements. The method can include measuring, by an electrocardiogram, one or more electrical activity measurements and expanding the expandable occluder based at least in part on the one or more electrical activity measurements.

A system of treating heart failure of a patient can include an expandable occluder. The expandable accumulator can include a first cap, a second cap, and a plurality of members. Each member can include a first end positioned on the first cap and a second end positioned on the second cap. The system can include a drive line attached to the first cap, the drive line configured to rotate the first cap with respect to the second cap in a first direction to cause the plurality of members to expand radially outward. The drive line can rotate the first cap with respect to the second cap in a second direction to cause the plurality of members to expand radially inward.

The system can include a drive motor connectable to the drive line, the drive motor configured to expand and collapse the expandable occluder. The system can include one or more implantable pressure sensors and an implantable control unit, the implantable control unit configured to receive pressure measurements from the one or more implantable pressure sensors, the implantable control unit configured to control the expandable occluder based on the pressure measurements. The system can include an elastic sheath covering the plurality of members. The plurality of members can be biased to expand radially outward.

### BRIEF DESCRIPTION OF THE DRAWINGS

Certain features of this disclosure are described below with reference to the drawings. The illustrated implementations are intended to illustrate, but not to limit, the implementations. Various features of the different disclosed implementations can be combined to form further implementations, which are part of this disclosure.

FIG. 1A illustrates cardiovascular features near the heart.

FIG. 1B illustrates the venous system in the chest.

FIG. 1C illustrates relevant anatomic structures in the upper chest.

FIG. 2A illustrates another view of relevant anatomic structures in the upper chest.

FIG. 2B illustrates an example of an implant location relative to the view shown in FIG. 2A.

FIG. 3A illustrates the pulmonary artery and azygos vein blood flow before intervention.

FIG. 3B illustrates an example of the blood flow of FIG. 3A after shunt implantation.

FIGS. 3C and 3D illustrate one example of the altered blood flow path in the pulmonary artery and azygos vein after implantation of an implementation of the shunt.

FIG. 4 illustrates an example of a system for treating heart failure of a patient.

FIG. 5 illustrates an example of insertion of a guidewire and other implantable devices.

FIG. 6A-6R illustrate a method of placing a shunt and an occluder system in the vasculature.

FIG. 7A illustrates an example of blood flow in the vasculature with the occluder system with a collapsed occluder of FIG. 6L.

FIG. 7B illustrates an example of blood flow in the vasculature with the occluder system with a collapsed occluder of FIG. 6L without the anchoring element.

FIG. 7C illustrates an example of blood flow in the vasculature with the occluder system with an expanded occluder of FIG. 6M.

FIG. 7D illustrates an example of blood flow in the vasculature with the occluder system with an expanded occluder of FIG. 6M without the anchoring element.

FIG. 8A illustrates an example of the occluder system with a collapsed occluder of FIG. 6L in a venous system.

FIG. 8B illustrates an example of the occluder system with an expanded occluder of FIG. 6M in a venous system.

FIG. 9A-9G illustrate examples of the occluder system in a venous system of FIG. 8A with sensors.

FIG. 10A shows a perspective view of an example of an occluder system with the occluder expanded.

FIG. 10B shows a perspective view of the example of the expanded occluder of FIG. 10A.

FIG. 10C shows a side view of the example of the expanded occluder of FIG. 10A.

FIG. 10D shows a perspective view of the example of the collapsed occluder of FIG. 10A.

FIG. 10E shows a cross-sectional front view of the example of the collapsed occluder of FIG. 10A.

FIG. 10F shows a cross-sectional front view of the example of the expanded occluder of FIG. 10A.

FIG. 10G shows a cross-sectional view of the example of the expanded occluder of FIG. 10A.

FIG. 11A illustrates an example of a collapsed occluder and a drive line detached from a drive motor.

FIG. 11B illustrates an example of an expanded occluder and a drive line detached from a drive motor.

FIG. 12 illustrates an example of a shunt connecting an azygos vein to a right pulmonary artery.

FIG. 13A-13E illustrate a method of placing an example of a shunt between an azygos vein and a right pulmonary artery.

FIG. 14A illustrates an example of a shunt.

FIG. 14B illustrates the example of the shunt of FIG. 14A secured to a right pulmonary artery.

FIGS. 15A-C illustrate an example of an occluder in an expanded state.

FIG. 16A-E illustrate the example of the occluder of FIG. 15A in various states of expansion.

### DETAILED DESCRIPTION

Various features and advantages of this disclosure will now be described with reference to the accompanying figures. The following description is merely illustrative in nature and is in no way intended to limit the disclosure, its application, or uses. This disclosure extends beyond the specifically disclosed implementations and/or uses and obvious modifications and equivalents thereof. Thus, it is intended that the scope of this disclosure should not be limited by any particular implementations described below. The features of the illustrated implementations can be modified, combined, removed, and/or substituted as will be apparent to those of ordinary skill in the art upon consideration of the principles disclosed herein. Furthermore, implementations disclosed herein can include several novel features, no single one of which is solely responsible for its desirable attributes or which is essential to practicing the systems, devices, and/or methods disclosed herein.

Parts, components, features, and/or elements of the systems and devices described herein that can function the same or similarly across various implementations are identified using similar reference numerals. Differences between the various implementations are discussed herein.

Implementations of the present application relate to controlling cardiac output to treat or prevent heart failure in patients. Certain embodiments are directed to creating a shunt (which may also be referred to as an implant, conduit or arteriovenous (AV) fistula) between the right pulmonary artery and the azygos vein to divert blood from the right pulmonary artery.

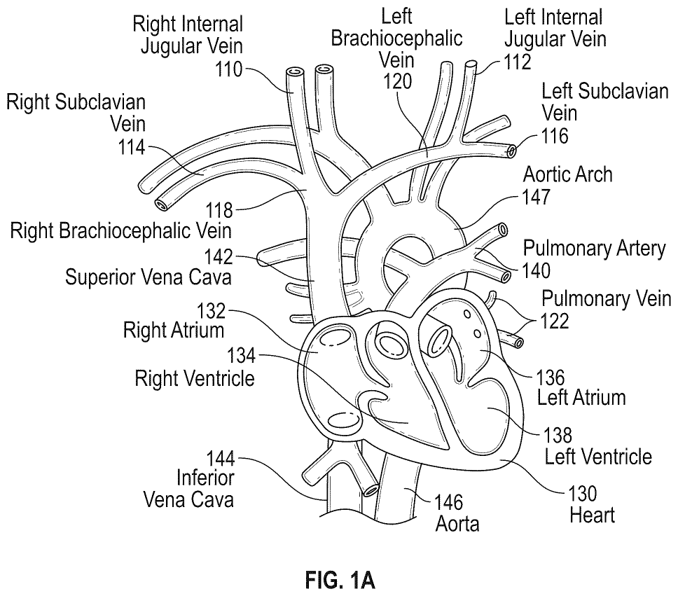

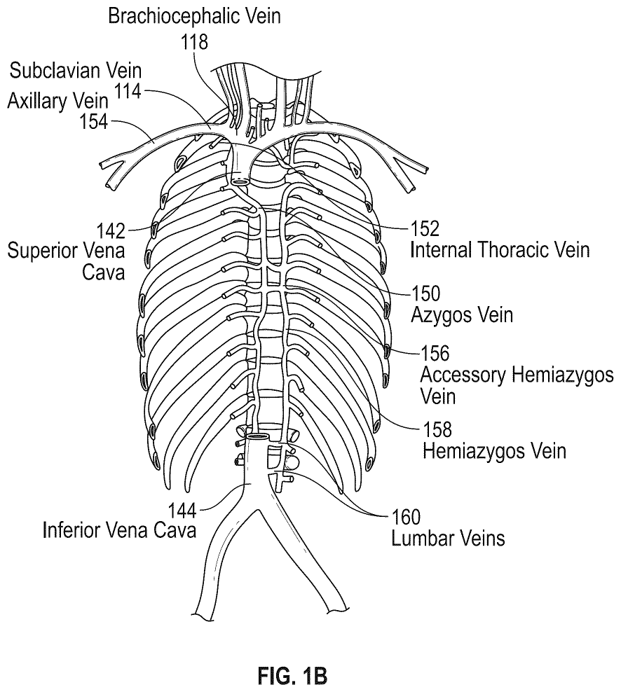

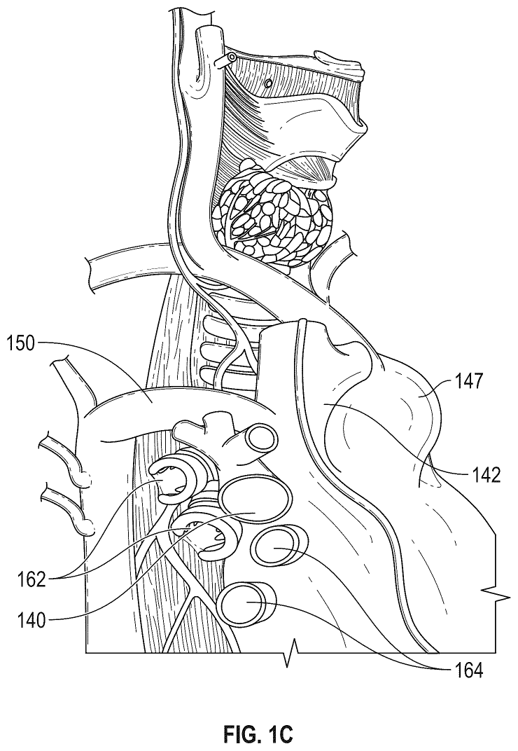

FIG. 1A illustrates a patient's anatomy including the heart 130 with the right atrium 132, the right ventricle 134, the pulmonary artery 140, the pulmonary veins 122, the left atrium 136, the left ventricle 138, and the aorta 146 including the aortic arch 147. The inferior vena cava 144 and superior vena cava 142 with the left internal jugular vein 112 and right internal jugular vein 110, left subclavian vein 116 and right subclavian vein 114, and left brachiocephalic vein 120 and right brachiocephalic vein 118 are also shown. This central cardiovascular complex collects deoxygenated blood from the body, pumps it to the lungs and back for oxygenation, and then pumps oxygenated blood to the body.

FIG. 1B illustrates a patient's venous anatomy in the chest, including the azygos vein 150 which flows into the superior vena cava 142, along with the accessory hemiazygos vein 156, the hemiazygos vein 158, the lumbar veins 160, and other vessels that flow into the azygos vein 150. Also shown are the right brachiocephalic vein 118, right subclavian vein 114, axillary vein 154, and the internal thoracic vein 152 that also flow into the superior vena cava 142.

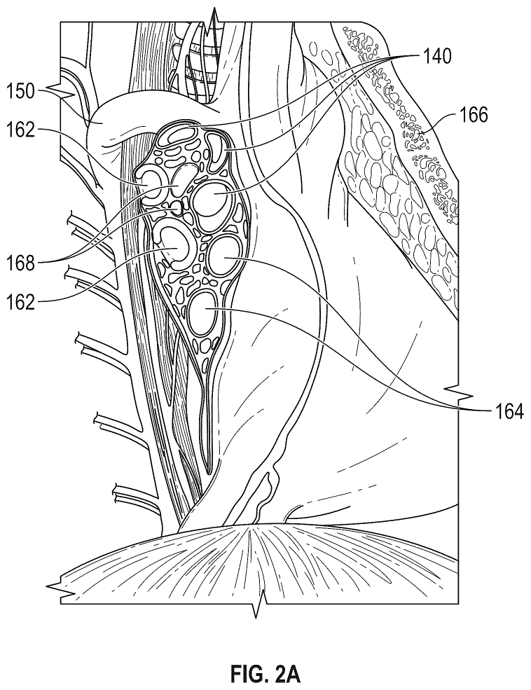

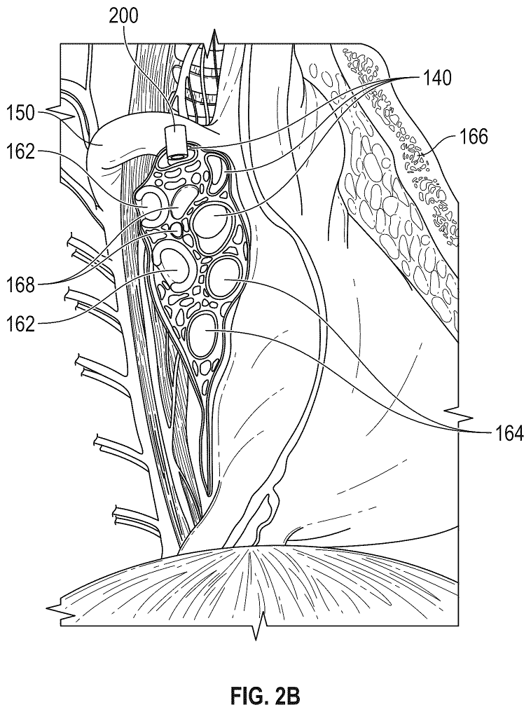

FIGS. 1C, 2A, and 2B illustrate the upper chest, including the sternum 166, right bronchus 162, right pulmonary veins 164, and lymph nodes 168. As shown in FIG. 2B, branches of the right pulmonary artery 140 extend adjacent the azygos vein 150, which provides for a possible location for implantation of a shunt 200. In some implementations, any branch of the pulmonary artery 140 may be suitable. For example, locations distal to the right pulmonary artery, including the truncus anterior, right superior trunk, apical artery, anterior artery, posterior recurrent artery, various intersegmentary branches, ascending artery, and interlobar artery may be suitable locations. Similarly, although the proximal arched section of the azygos vein 150 that extends over the pulmonary artery 140 just before the junction with the superior vena cava 142 is shown, any location along the azygos vein 150 may be suitable. Implant location may be chosen based on the patient's particular anatomy, the distance between the branch of the pulmonary artery 140 and the azygos vein 150, the estimated blood flow through the respective vessels, estimated pressure differentials, the ease of surgical access, avoidance of any intervening structures (such as right bronchus 162, lymph nodes 168, nerves, and other non-target vasculature), and/or the preference of the implanting physician, among other factors.

A device, such as shunt 200, implanted at this location in some implementations is suitable for redirecting or diverting an amount of blood from the right pulmonary artery 140 that is leaving the right ventricle 134 of the heart 130 into the azygos vein 150 that passes adjacent to the right pulmonary artery 140. In some implementations, shunt 200, shown within both the pulmonary artery 140 and azygos vein 150 for clarity in FIG. 2B, may form a passage between the arterial and venous systems to help create a splanchnic compartment in the thoracic vessels. When the right pulmonary artery 140 is connected to the azygos vein 150, the pressure differential between the arterial and venous systems may cause blood to flow from the pulmonary artery 140 into the azygos vein 140, and further may create backflow in the azygos vein 150. An implant such as the shunt 200 may be delivered percutaneously into a patient in a collapsed configuration and expanded to an expanded configuration upon implantation.

The blood that is shunted from the right pulmonary artery 140 into the azygos vein 150 may cause the intercostal veins to grow larger (dilate) and pressurize, which effectively increases intravascular volume and creates a tank of extra blood within the intercostal veins. The redirecting or diverting of blood may be sufficiently accommodated in a venous capacitance system. The redirecting or diverting of blood may result in increasing venous capacitance to increase cardiac output.

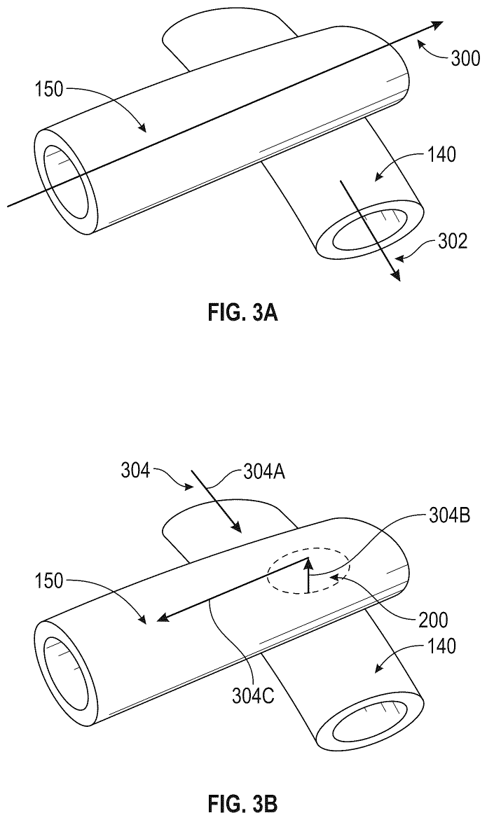

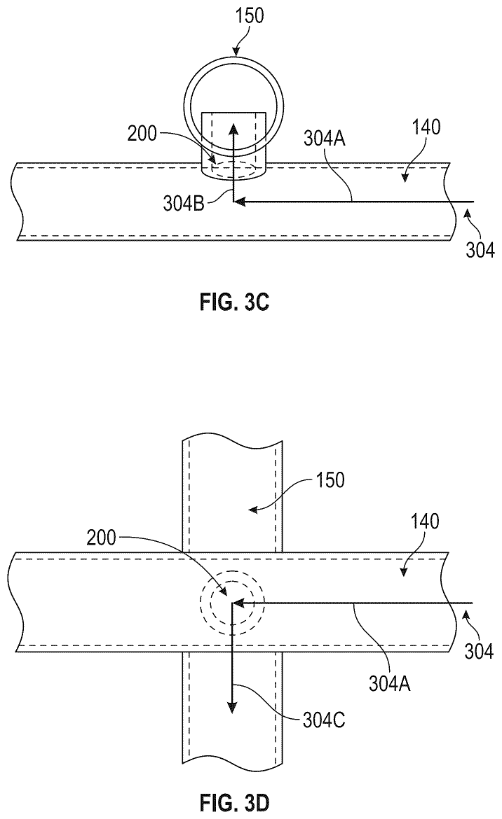

As illustrated in FIG. 3A, the azygos vein 150 normally flows in a forward or antegrade direction 300 into the superior vena cava 142. The pulmonary artery 140 flows in a forward or antegrade direction 302 to the lungs. As illustrated in FIG. 3B, creation of a passage or fistula 200 between the pulmonary artery 140 and the azygos vein may cause a diversion or redirection of blood flow in the ayzgos vein. The passage or fistula 200 may be provided by any of the implants as described herein or may be created by other devices or techniques. In some implementations, after creation of the passage or fistula 200, blood from the right ventricle 134 flows through the pulmonary artery 140 along forward or antegrade direction 304A, as normal. Blood may then be redirected along direction 304B, through passage 200, and into the azygos vein 150. In some implementations, the connection between the azygos vein 150 and the superior vena cava 142 may be at least partially or fully occluded to direct the blood that has been diverted from the right pulmonary artery 140 in a retrograde direction 304C into the accessory hemiazygos vein 156, the hemiazygos vein 158, the internal thoracic veins 152, or other vessels including the intercostal veins, the internal mammary veins, and others as described above.

The amount of blood that is diverted from the right pulmonary artery 140 into the azygos vein 150 reduces the amount of blood that reaches the lungs, which may advantageously reduce the stress and decongest the lungs and reduce left ventricular end diastolic pressure (LVEDP). The diverting of blood may be sufficient to reduce pulmonary artery pressure to relieve pulmonary hypertension and consequently reduce a workload of a right ventricle 134 of the patient. Treatment can be tailored by controlling one or both of the amount of blood diverted from the right pulmonary artery 140 and the amount of blood that flows from the azygos vein 150 to the superior vena cava 142.

In some implementations, such as the example shown in FIGS. 3C-3D, the implant may be a short shunt 200 configured to provide a connection between two vessels (e.g., between the pulmonary artery 140 and the azygos vein 150). This shunt 200 may preferably be positioned between the pulmonary artery 140 and the azygos vein 150 without extending substantially into the lumen of either vessel. As illustrated in side-view FIG. 3C, the shunt 200 may be implanted to create a flow pathway between a branch of the pulmonary artery 140 and the azygos vein 150. This location allows blood flow 304 to continue along forward or antegrade direction 304A within the branch of the pulmonary artery 140 toward the lung, as normal, and a portion of the blood flow 304 to be diverted through the shunt 200 along direction 304B into the azygos vein 150. As illustrated in top view FIG. 3D, when antegrade flow to the superior vena cava 142 is at least partially or fully occluded, blood flow 304 exits the shunt 200 into the azygos vein 150 and may provide backflow along reverse or retrograde direction 304C. Such backflow may create a thoracic tank in the chest vessels, such as intercostals, the accessory hemiazygos vein 156, the hemiazygos vein 158, and others as discussed above. A portion of the diverted blood may continue to flow in an antegrade direction toward the superior vena cava 142. As described further below, in some implementations the shunt 200 may comprise a passive valve. Also as described further below, in some implementations blood flow from the azygos vein 150 to the superior vena cava 142 may be separately restricted or occluded, such as by positioning an additional flow restricting implant within the azygos vein 150 downstream of the shunt 200.

Diversion of blood in the azygos vein 150 in this and other implementations may advantageously create a thoracic tank that can mimic a splanchnic vascular capacitance and redistribute blood into a splanchnic compartment, thereby offloading the heart. Reducing pulmonary artery pressure can relieve pulmonary hypertension and consequently reduce a workload of a right ventricle. Diverting the blood flow may also decongest lungs of the patient and reduce a left ventricular end diastolic pressure (LVEDP).

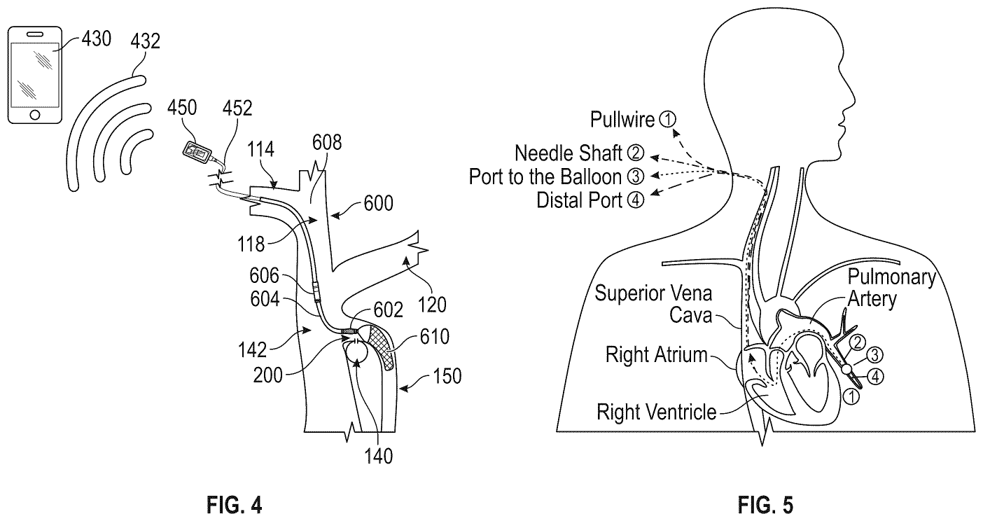

FIG. 4 illustrates an example of a system for treating heart failure of a patient. The system can include an occluder system 600 and/or a shunt 200. The occluder system 600 can include a control unit 450. The control unit 450 can be in wireless communication 432 with an external device 430.

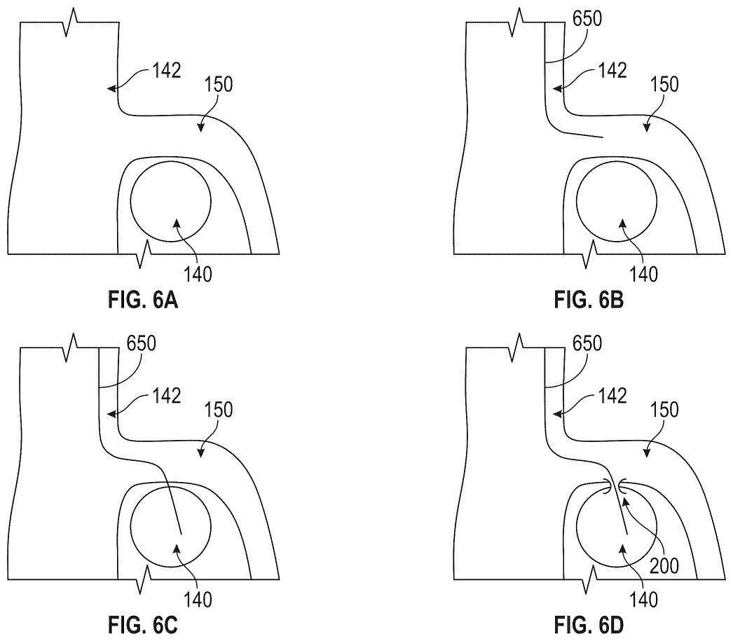

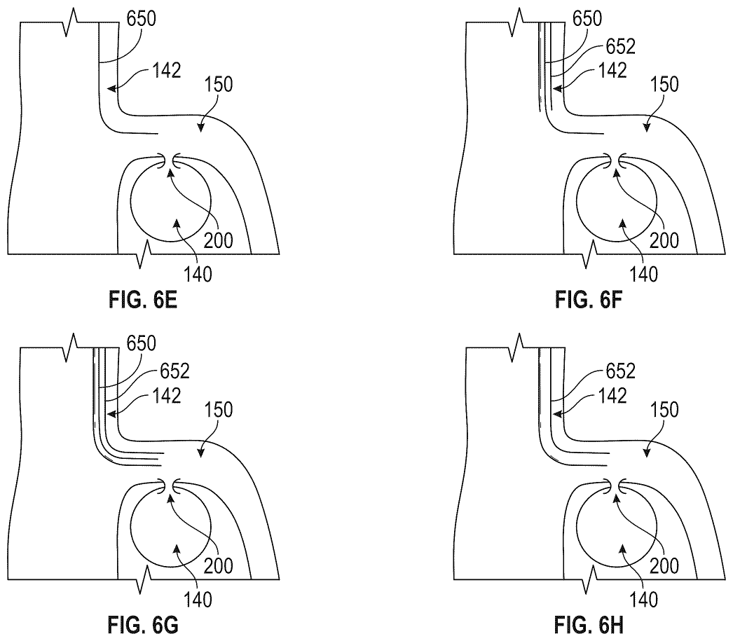

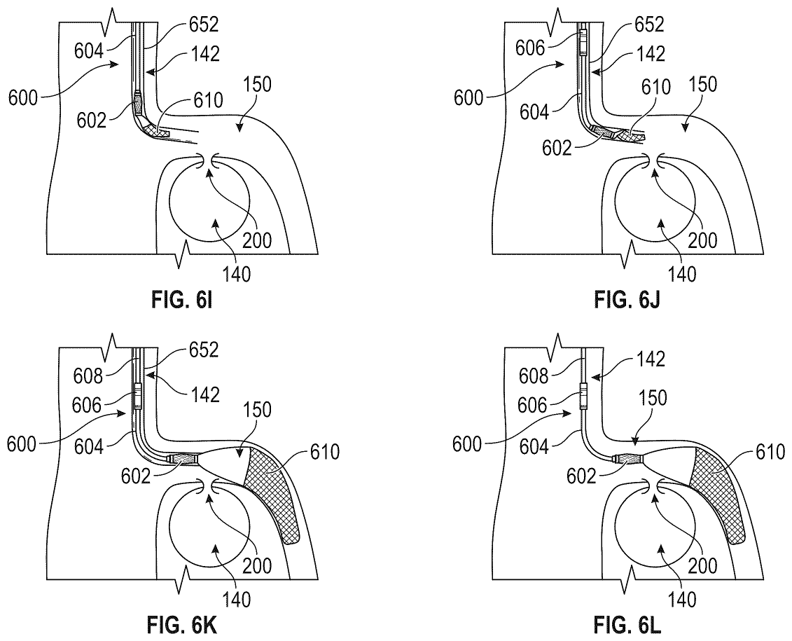

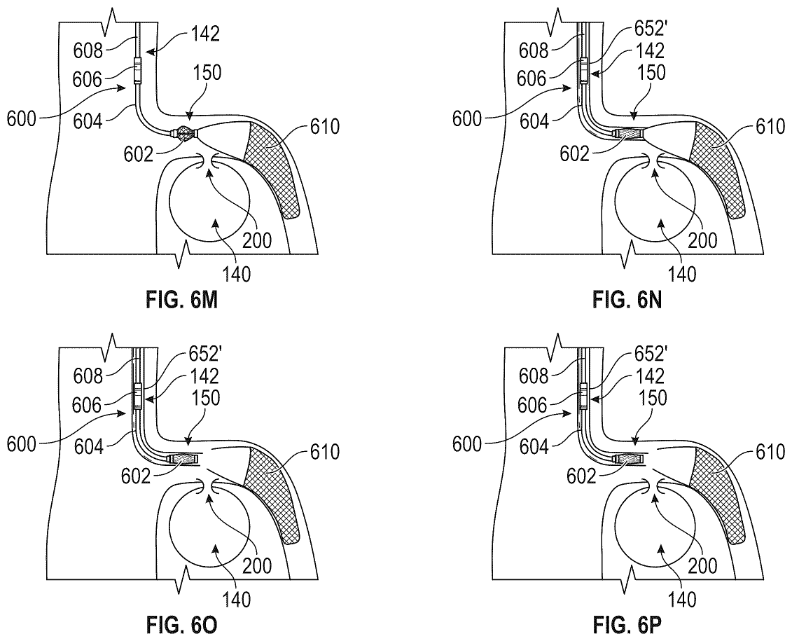

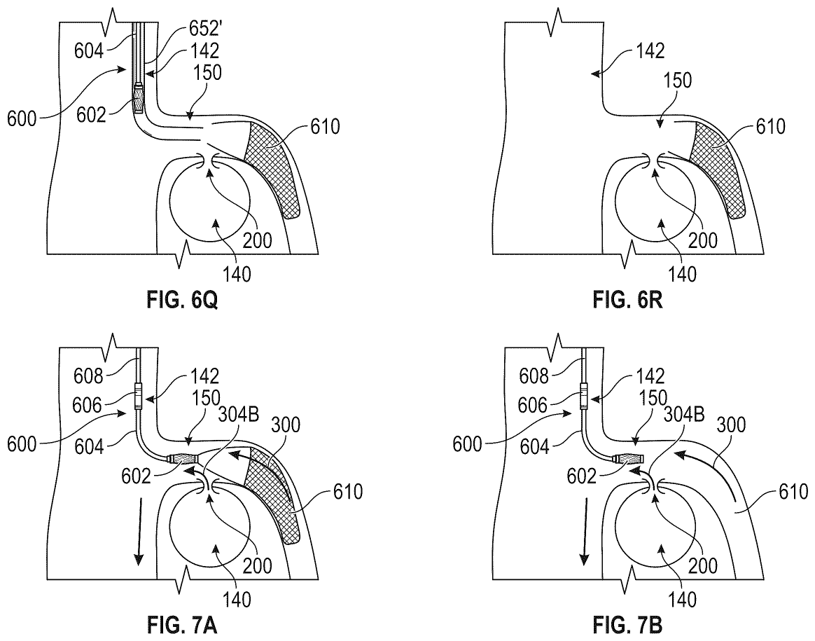

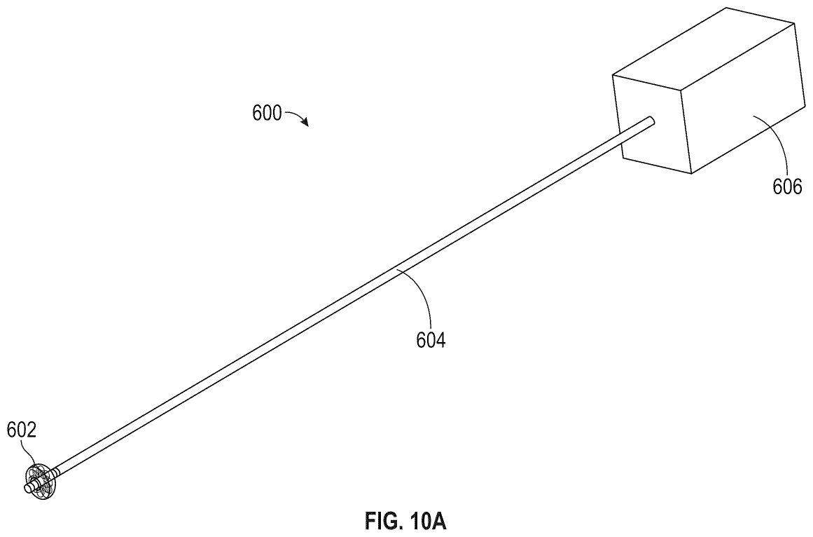

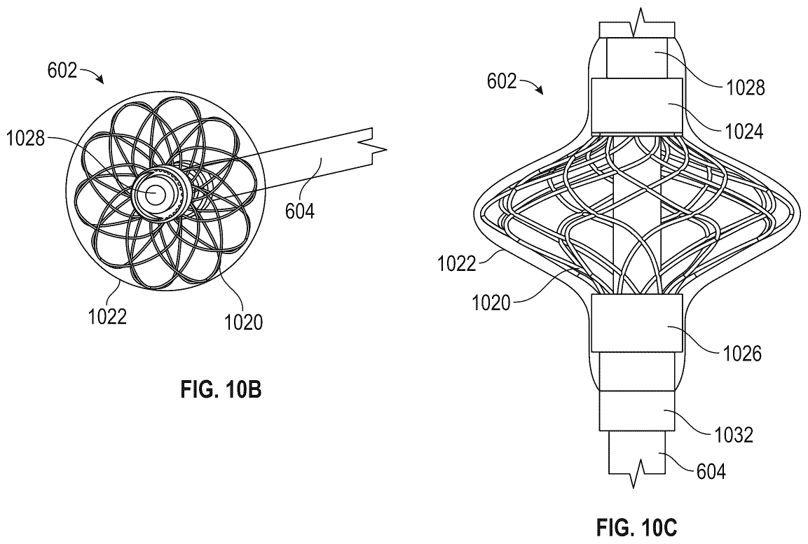

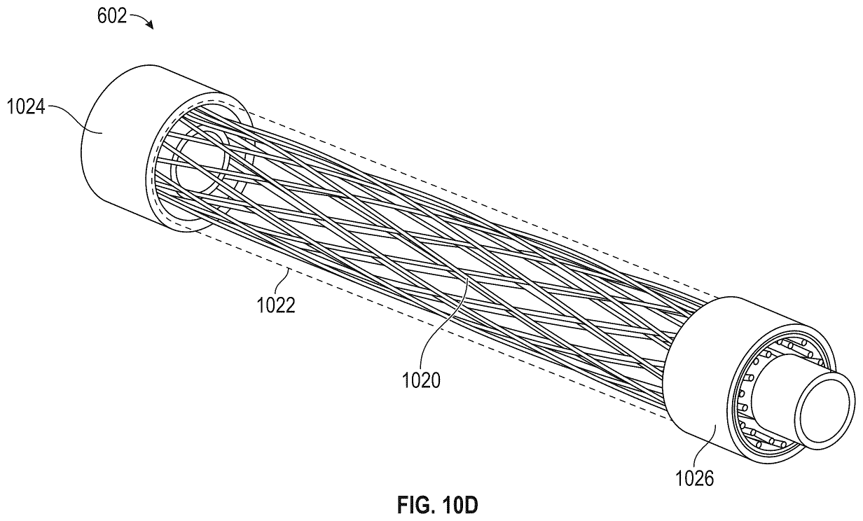

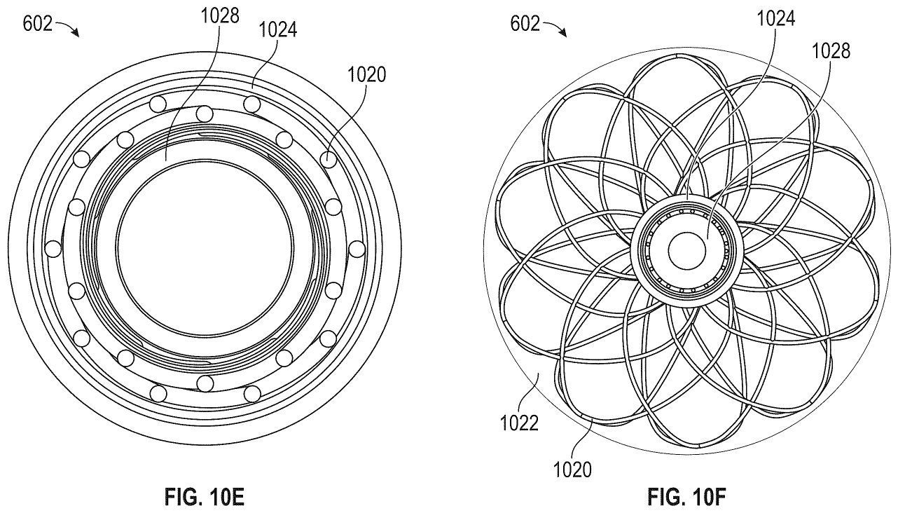

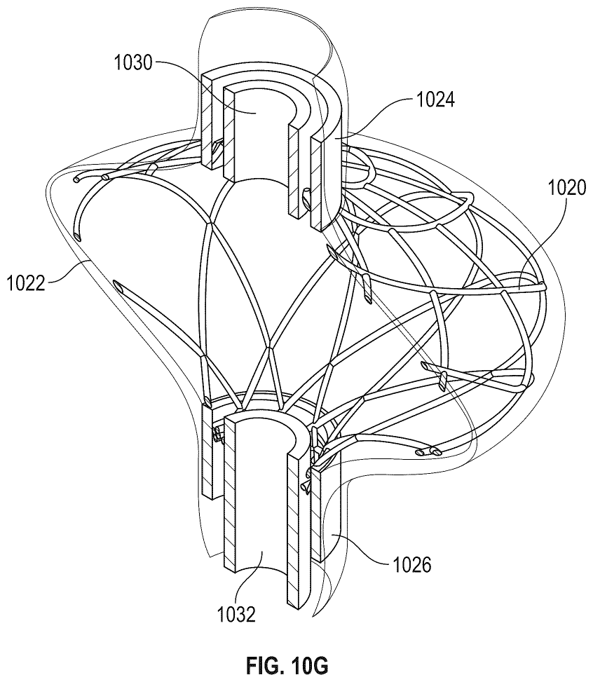

As illustrated, a distal portion of the occluder system 600 can be positioned in the azygos vein 150. The control unit 450 can be subcutaneously implanted in the chest, for example near the collar bone. In this configuration, the occluder system 600 can extend through the superior vena cava 142. The occluder system 600 can extend through the right brachiocephalic vein 118 and/or the right subclavian vein 114. The location of the various elements may change depending on the location of distal portion of the occluder system 600 and the control unit 450. The occluder system 600 can at least partially occlude blood flow through the vein as described in FIGS. 6A-6R and 10A-10G.

In some implementations, the diverting of blood is sufficient to decongest lungs of the patient and reduce a left ventricular end diastolic pressure (LVEDP) and/or to reduce pulmonary artery pressure to relieve pulmonary hypertension and consequently reduce a workload of a right ventricle of the patient. In particular, occluding blood flow from the azygos vein 150 to the superior vena cava 142 can have an acute effect on pulmonary hypertension. In some implementations, the diverting of blood is sufficient to mimic a splanchnic vascular capacitance. In some implementations, the diverting of blood is sufficient to cause dilation and/or increased pressure within intercostal veins of the patient. In some embodiments, the automatic control of the flow of the diverted blood using the occluder system 600 can be used optimize a patient's LVEDP, pulmonary artery pressure, central venous pressure, and other physiological parameters.

The occluder system 600 may include a control unit 450 configured to activate the occluder 602. The control unit 450 may be in wired connection with the occluder 602 via a controller connection wire 452. The controller connection wire 452 can connect to a lead 608 of the occluder system. In some embodiments, the controller connection wire 452 can connect directly to a drive motor 606. The drive motor can mechanically expand and/or collapse the occluder 602. In other configurations, the control unit 450 can control the occluder system 600 wirelessly, for example by wirelessly controlling the drive motor 606.

The control unit 450 can be a Controller Area Network (CAN) bus. The control unit 450 can be implantable. For example, the control unit 450 can be implanted near the collar bone. The control unit 450 can regulate expansion of the occluder 602 of the occluder system 600. The control unit 450 can include batteries and/or a wireless charging coil. The control unit 450 can charge wirelessly while implanted in the body through inductive charging. The control unit 450 can house the battery for the drive motor 606, the pressure sensors, and other electronics.

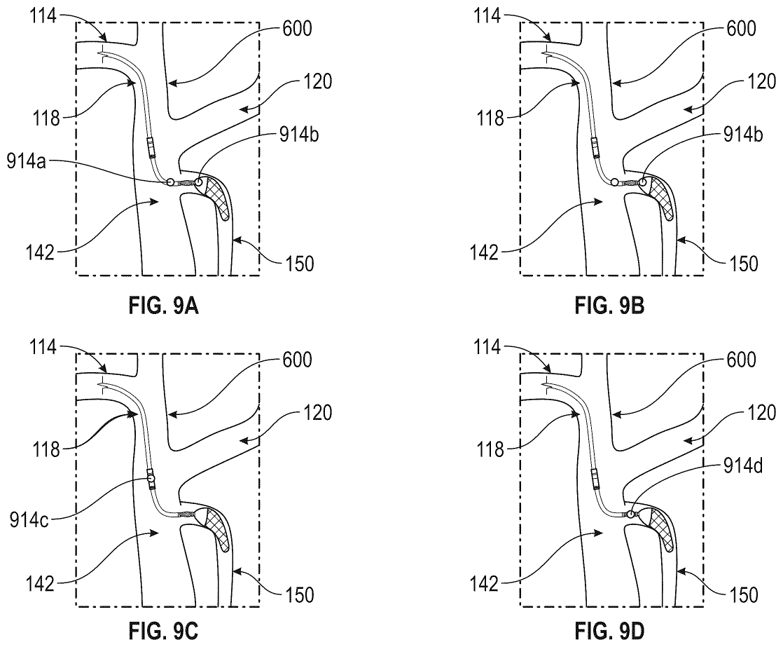

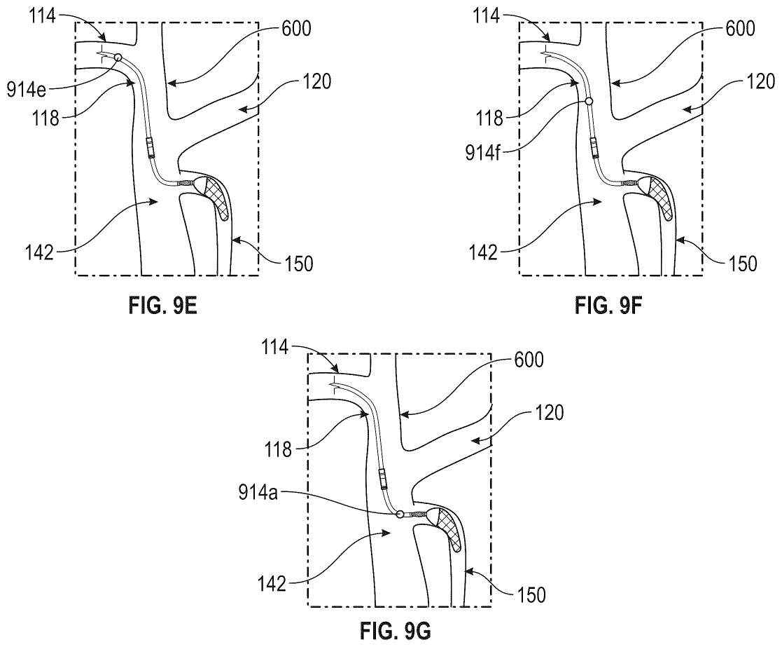

The control unit 450 can receive feedback or measurements, from one or more sensors. The sensors can be implantable. As a non-limiting example, the one or more sensors can include pressure sensors, electrocardiograms, and/or accelerometers. For example, the control unit 450 can include an electrocardiogram and/or an accelerometer. One or more pressure sensors can be positioned along the occluder system 600 as described below with respect to FIG. 9A-9G. In some embodiments, any of the sensors can be positioned in the vasculature independent of the occluder system 600. The pressure sensors can measure right ventricle pressure, central venous pressure, aortic pressure, left atrial pressure, azygos vein pressure, right pulmonary artery pressure, superior vena cava pressure, and/or pressure in another vessel. The sensors can communicate wirelessly with the control unit 450 or through the controller connection wire 452.

The control unit 450 can control the occluder 602 at least in part based on feedback from the one or more sensors, for example pressure measurements from the pressure sensors. In some embodiments, the control unit 450 can automatically control the occluder 602 based on feedback from the sensors. In other embodiments, the control unit controls the occluder 602 based on user activation.

In some configurations, the control unit 450 controls the occluder 602 based at least in part on activity measurements from the accelerometer or electrical activity measurements from the electrocardiogram. The electrocardiogram and accelerometer can allow the control unit 450 to more accurately measure pressure, for example by taking into account a patient's standing or supine position, level of exercise, or other factors that affect pressure in the vessels. For example, the control unit 450 can increase or decrease the thresholds described below based on the factors determined by the electrocardiogram and/or the accelerometer.

In some implementations, the control unit 450 can cause the occluder 602 to expand or collapse at least in part based on a measured or derived pressure measurement. For example, the control unit 450 can cause the occluder 602 to expand or collapse depending on whether the pressure measurement is above or below a threshold. For example, the control unit 450 can determine central venous pressure based on the pressure measurements collected from the one or more sensors. The control unit 450 can cause the occluder 602 to expand when central venous pressure, is above an upper threshold. The upper threshold can be 15 mmHg. In some embodiments, the upper threshold can be a value of at least 10 mmHg, for example between 10 mmHg and 20 mmHg. In some embodiments, the upper threshold can be at least about 6 mmHg, for example between 6 mmHg and 30 mmHg. The control unit 450 can cause the occluder 602 to collapse when central venous pressure is below a lower threshold. The lower threshold can be 5 mmHg. In some embodiments, the lower threshold can be less than or equal to 10 mmHg, for example between 3 mmHg and 10 mmHg. In some embodiments, the lower threshold can be less than or equal to 15 mmHg, for example between 1 mmHg and 14 mmHg. The control logic, including the feedback loop, may be optimized to treat heart failure patients, for example by controlling the amount of blood that is diverted from the right pulmonary artery 140 into the azygos vein 150. This may reduce the amount of blood that reaches the lungs, which may advantageously reduce the stress and decongest the lungs and reduce left ventricular end diastolic pressure (LVEDP). This may also cause the pressure in the right pulmonary artery 140 to equalize.

The control unit 450 can include a wireless transceiver for communication with external devices 430. The control unit 450 can engage in wireless communication 432 with an external device 430, for example a computer, smart phone, other smart device, or database. In some embodiments, the control unit 450 can control the occluder system 600 based on user input from an external device. For example, the occluder 602 may be activated remotely, via a Bluetooth connection, via a smartphone app. The control unit 450 can send pressure data, performance data, battery health, and other data to external devices 430, for example to a patient or physician's devices. The control unit 450 can upload the data to a database or cloud in a constant manner. The system can use a neural network to optimize an algorithm for expanding and collapsing the occluder.

The occluder system 600 and control unit 450 can be chronically implanted. In some embodiments, the control unit 450 can be external to the patient, for example during a procedure. The occluder system 600 can be temporarily inserted in a patient with the control unit 450 external to the patient in order to temporarily occlude blood flow.

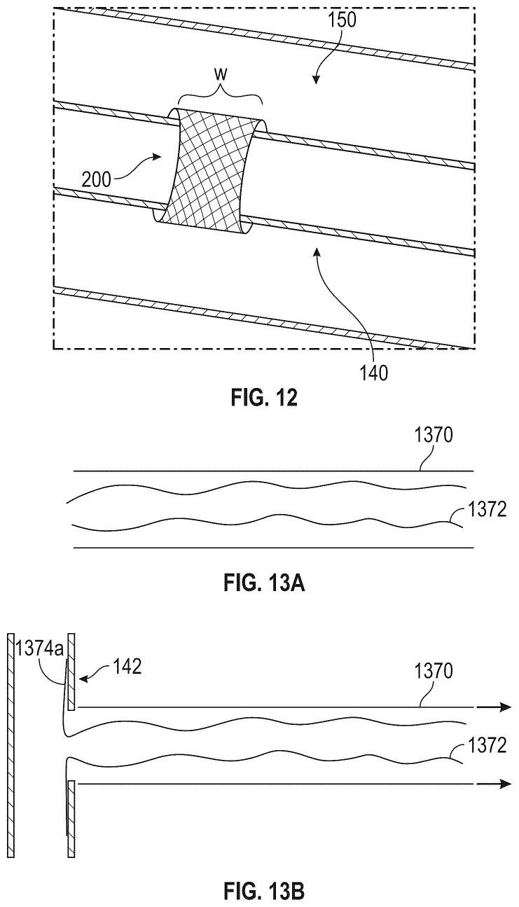

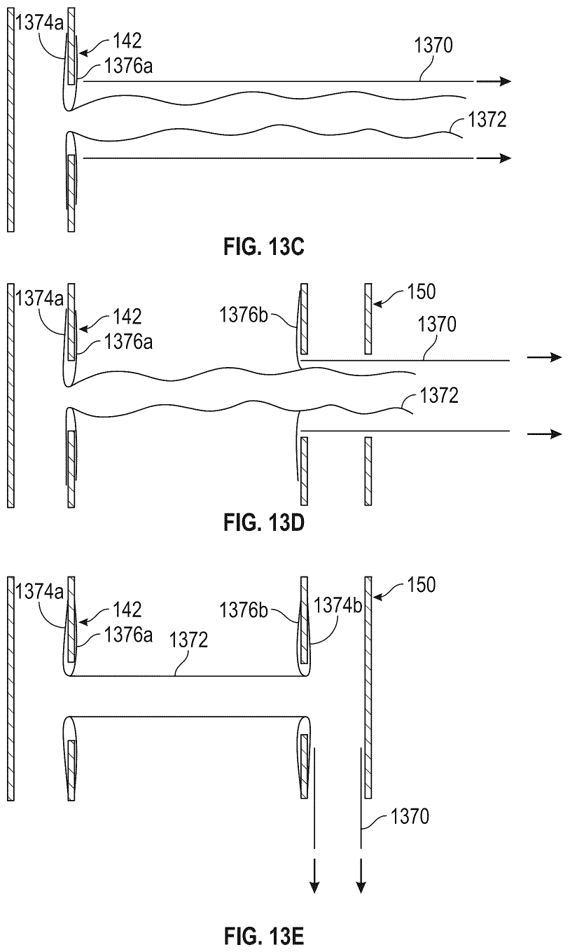

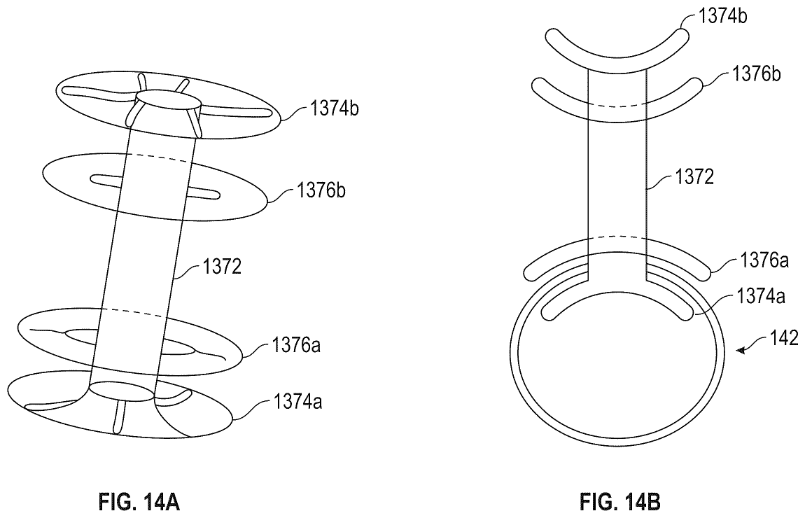

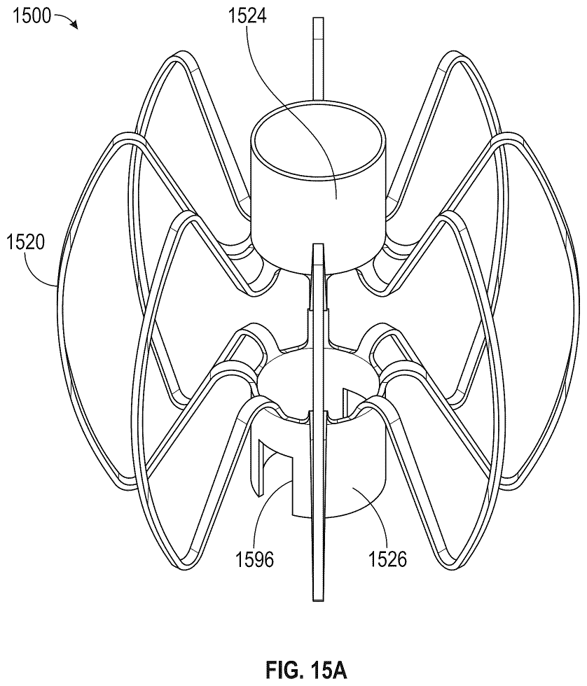

The shunt 200 can be positioned between the azygos vein 150 and the right pulmonary artery 140. The shunt 200 can direct blood flow from an artery to a vein as described in FIGS. 6D-6R and 12. The shunt 200 can passively allow blood flow from the artery to the vein. In some embodiments, the shunt 200 can include a check valve, as described with respect to FIG. 12. In some embodiments, the shunt can be similar to the shunt described with respect to FIGS. 13A-13E and 14A-14B.

FIG. 5 illustrates an example of insertion of a guidewire 650 and other implantable devices. The guidewire 650 may be placed using various methods including those described in U.S. Provisional Application No. 63/591,376, filed on Oct. 28, 2023, and U.S. Non-Provisional application Ser. No. 18/918,681, filed on Oct. 17, 2024, and both of which are hereby incorporated by reference in their entireties.

In some implementations, the azygos vein 150 may be accessed via the superior vena cava 142. In some implementations, the azygos vein 150 may be accessed via the inferior vena cava 144. In some implementations, a Swan-Ganz approach may be used to access the pulmonary artery 140. For example, the pulmonary artery 140 may be accessed via the superior vena cava 142 or the inferior vena cava 144, through the right atrium 132 and the right ventricle 134, and into the pulmonary artery 140. In some implementations, the azygos vein 150 and pulmonary artery 140 are accessed separately, and each end of the implant may be separately deployed and/or placed via tools in each vessel. In some implementations, the pulmonary artery 140 and azygos vein 150 are accessed from one vessel (e.g., a Swan-Ganz approach through the pulmonary artery 140 is used to create an opening to the azygos vein 150, or the reverse), and an implant may be placed via tools in the access vessel. Access via the superior vena cava 142 can allow the control unit 450, as described with respect to FIG. 4, to be implanted subcutaneously. The controller connection wire 452 can connect the control unit 450 to the occluder system 600 through the access point on the superior vena cava 142.

FIG. 6A illustrates a vasculature of a patient including a superior vena cava 142, an azygos vein 150, and a right pulmonary artery 140. FIG. 6B illustrates an example of a guidewire 650 in the vasculature of FIG. 6A. FIG. 6C illustrates an example of a guidewire 650 in the vasculature of FIG. 6A extending through a wall of the azygos vein 150 and a wall of the right pulmonary artery 140. FIG. 6D illustrates an example of a shunt 200 positioned in the vasculature of FIG. 6A along a guidewire 650.

As shown in FIGS. 6B-6C, a guidewire 650, or venous wire, can enter from above the superior vena cava 142, for example from the internal jugular veins, common femoral veins, subclavian veins, basilic vein, and/or brachial veins. A proximal end of the guidewire 650 can be outside the body. The guidewire 650 can be controlled manually or using robotic guidance. The guidewire 650 can be inserted through a wall of the azygos vein 150 and into a wall of the right pulmonary artery 140, for example using a needle. In other implementations, the guidewire 650 may be passed from the right pulmonary artery 140 to the azygos vein 150. In other implementations, the guidewire 650 may be passed from the inferior vena cava 144 to the right atrium 132 to the superior vena cava 142, for example using transfemoral venous access. One end or both ends of the guidewire 650 can be external to the patient.

In some implementations, magnets may be used to align two catheters to facilitate delivery of the guidewire 650 from one vessel to the other. The guidewire 650 may be used to enable access for a delivery device used to deliver the shunt 200 and/or the occluder system 600. In some implementations, the magnets may be connected to control wires. In some implementations, control wires may be used to switch a polarity of the magnets to selectively repel, which may be useful for removal of the catheters after delivery. In some implementations, magnets may be electromagnets, and control wires may be used to selectively activate and deactivate the magnets at different times during placement, delivery and/or removal of the catheters. Other methods are described in U.S. Provisional Application No. 63/591,376, filed on Oct. 18, 2023, and U.S. Non-Provisional application Ser. No. 18/918,681, filed on Oct. 17, 2024, and both of which are hereby incorporated by reference in their entireties. In some implementations, the guidewire 650 can enter from the right pulmonary artery 140 to the azygos vein 150.

As shown in FIG. 6D, the shunt 200 can be positioned between the azygos vein 150 and right pulmonary artery 140 along the guidewire. The shunt 200 can expand when it reaches its target location. The shunt 200 can allow flow between the artery and the vein. The shunt can be similar to the shunt 200 of FIGS. 2B, 3C, 3D, 4, 12, 13A-13E and/or 14A-14B. After the shunt 200 is implanted, the guidewire may be retracted from the right pulmonary artery 140 (FIG. 6E) for use delivering the occluder system 600.

FIG. 6E illustrates an example of the guidewire 650 retracted from the right pulmonary artery 140 so a distal end of the guidewire 650 is in the azygos vein 150. FIGS. 6F and 6G illustrate a guide sheath 652 being inserted over the guidewire 650. FIG. 6H illustrates the guidewire 650 removed from the vasculature with the guide sheath 652 positioned in the vasculature. FIGS. 6I, 6J, and 6K illustrate the occluder system 600 being inserted into the vasculature through the guide sheath 652. FIG. 6L illustrates the guide sheath 652 removed from the vasculature with the occluder system 600 positioned in the vasculature.

As shown in FIGS. 6F and 6G, the guide sheath 652 can be tracked over the guidewire 650 and positioned with a distal end of the guide sheath 652 in the azygos vein 150. The guide sheath 652 can be a hollow tube. The guide sheath 652 can be positioned with a distal end of the guide sheath 652 proximal to an outlet of the shunt 200. In other embodiments, the guide sheath 652 can be positioned with a distal end of the guide sheath 652 distal to an outlet of the shunt 200. One end of the guide sheath 652 can be external to the patient.

As shown in FIGS. 6H and 6I, the occluder system 600 can be inserted through the guide sheath 652. It can be advantageous to deliver the occluder system 600 through the guide sheath 652 because the occluder 602 may have a closed distal end, which can make using a guide sheath preferable to using a guidewire. The distal end of the occluder 602 may be closed to prevent blood from flowing into the occluder 602. The anchoring element 610, or expandable stent, can be compressed in the guide sheath 652 as the occluder system 600 is being positioned in the vasculature to prevent the anchoring element 610 from contacting a vessel wall before placement in the azygos vein 150. In some embodiments, an anchoring element 610 that is a J-tip can be delivered through the guide sheath 652 either compressed or uncompressed. In some embodiments, the occluder system 600 can be anchored in the superior vena cava 142 and/or the ostium of the azygos vein 150. In some embodiments, the occluder system 600 can be positioned using a guidewire and the occluder 602 can be sealed after placement.

As shown in FIGS. 6H and 6I, the occluder system 600 can enter from above the superior vena cava 142, for example from the internal jugular veins, common femoral veins, subclavian veins, basilic vein, and/or brachial veins. The occluder 602 can be positioned in the azygos vein 150, as seen in FIG. 6K. In some embodiments, the occluder 602 can be positioned at least partially in the superior vena cava 142. In some embodiments, the occluder system 600 can enter from below the superior vena cava 142, for example from the right atrium and/or inferior vena cava using transfemoral venous access. In some implementations, the occluder system 600 can enter from the right pulmonary artery 140 to the azygos vein 150.

As shown in FIGS. 6K and 6L, the occluder 602 can be positioned in the azygos vein 150. The drive line 604 and the drive motor 606 can be positioned in the superior vena cava 142. A distal end of the lead 608 can be positioned in the right subclavian vein 114 and connect to a control unit 450 as described in FIGS. 5, 8A, and 8B.

As shown in FIGS. 6K and 6L, the anchoring element 610 can be positioned in the azygos vein 150 distal of the occluder 602 to secure the occluder system 600 in place. The anchoring element 610 can expand after exiting a distal end of the guide sheath 652. The anchoring element 610 can engage a vessel wall, for example the inner walls of the azygos vein 150, to prevent the occluder system from moving significantly. The anchoring element 610 can be a stent, for example an expandable stent, or a J-tip. The anchoring element 610 can be connected to the occluder 602 by a plurality of anchoring wires. The anchoring element 610 can allow collateral flow. Advantageously, the anchoring element 610 can be positioned in a vessel without impeding flow through the vessel. The anchoring element 610 can be detached and left in the vessel without impeding vessel function. In some embodiments, the anchoring element 610 can be deployed once the occluder 602 is in the azygos vein 150. The anchoring element 610 can be removably connected to the occluder system 600 as described in FIGS. 6N-6R.

FIG. 6J illustrates an example of an occluder system 600 with an expanded occluder 602 and anchoring element 610 in the azygos vein 150 in the vasculature. The occluder 602 can expand as described with respect to FIG. 10A-10G. As shown in FIG. 6J, the expanded occluder 602 can completely occlude blood flow by contacting the walls of the vessel, for example the walls of the azygos vein 150. In some embodiments, the expanded occluder 602 can at least or only partially occlude blood flow without contacting a wall of the vessel, for example a wall of the azygos vein. The expandable cage 1020 and/or the elastic sheath 1022 (FIG. 10A-10G) of the expanded occluder 602 can prevent blood from flowing in a certain direction, for example antegrade from the azygos vein 150 to the superior vena cava 142. The expanded occluder 602 can occlude 100% of blood flow from the azygos vein 150 to the superior vena cava 142. In some embodiments, the expanded occluder 602 can prevent at least 60% or between 60% and 100% of blood flow from the azygos vein 150 to the superior vena cava 142. In some embodiments, the expanded occluder 602 can prevent at least 25% or between 25% and 100% of blood flow from the azygos vein 150 to the superior vena cava 142. The occluder 602 can expand for approximately 30 seconds before collapsing. In some embodiments, the occluder 602 can expand for approximately 20-40 seconds before collapsing. In some embodiments, the occluder 602 can expand for approximately 1 second to 10 minutes before collapsing.

In some instances, it may be desirable to remove the occluder system 600 from the vasculature. FIG. 6N illustrates an example of a guide sheath 652′ being inserted into the vasculature to remove the occluder system 600 from the vasculature. FIG. 6O illustrates an example of an occluder system 600 detached from the anchoring element 610 in the vasculature. FIGS. 6P and 6Q illustrate an example of the occluder system 600 detached from the anchoring element 610 exiting the vasculature. FIG. 6R illustrates an example of an anchoring element 610 in the vasculature.

As shown in FIG. 6N, the anchoring element 610 can be removably connected to the occluder system 600. This can be advantageous as the anchoring element 610 may be difficult to remove from the vessel once it is endothelialized. The occluder system 600 can still be removed, as it is less likely to be endothelialized, as it can avoid making significant contact with a wall of the vessel. Since the distal anchoring element 610 is likely to be fully in contact with the wall of the azygos vein 150 for an extended period of time, it is unlikely to be able to be removed percutaneously. The anchoring element 610 can be detached from the occluder system 600 when the occluder system 600 is removed from the vasculature.

The anchoring element 610 can be detached using a cutting tool or cautery tool. For example, the cutting tool can be used to detach the anchoring element 610 through the guide sheath 652′. In other implementations, the anchoring wires may be mechanically released from the occluder 602.

As shown in FIGS. 6P and 6Q, the occluder system 600 can be removed by retracting it upward from the superior vena cava 142, for example from the internal jugular veins, common femoral veins, subclavian veins, basilic vein, and/or brachial veins, using the guide sheath 652.

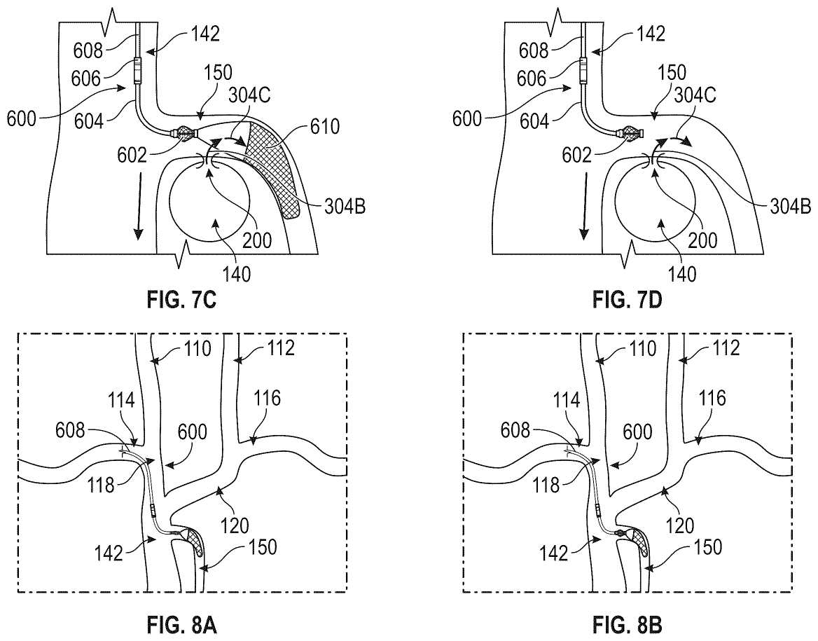

FIG. 7A illustrates an example of blood flow in the vasculature with the occluder system 600 with a collapsed occluder 602 of FIG. 6I. FIG. 7B illustrates an example of blood flow in the vasculature with the occluder system 600 with a collapsed occluder 602 of FIG. 6I with the anchoring element removed for clarity. FIG. 7C illustrates an example of blood flow in the vasculature with the occluder system 600 with an expanded occluder 602 of FIG. 6J. FIG. 7D illustrates an example of blood flow in the vasculature with the occluder system 600 with an expanded occluder 602 of FIG. 6J with the anchoring element removed for clarity.

As shown in FIGS. 7A-7B, with the occluder 602 collapsed, blood can flow from the right pulmonary artery 140 through the shunt 200 to the azygos vein 150 along direction 304B. Blood can flow in a forward or antegrade direction 300 from the azygos vein 150 to the superior vena cava 142. In some embodiments, some blood can flow in the reverse or retrograde direction in the azygos vein 150. Blood can flow from the azygos vein 150 toward the superior vena cava 142 without significant impedance because the collapsed occluder 602 does not block a significant portion of the ostium of the azygos vein 150. Blood can flow from the superior vena cava 142 to the right atrium 132.

As shown in FIGS. 7C-7D, with the occluder 602 expanded, blood can flow from the right pulmonary artery 140 through the shunt 200 to the azygos vein 150 along direction 304B. Blood can flow in the reverse or retrograde direction 304C in the azygos vein 150. Blood is occluded from flowing from the azygos vein 150 toward the superior vena cava 142. Occlusion of flow toward the superior vena cava 142 can cause more blood to flow in the reverse or retrograde direction 304C in the azygos vein 150. The flow of blood from the azygos vein 150 to the superior vena cava 142 can be completely stopped by the expanded occluder 602. In some embodiments, some blood can flow from the azygos vein 150 to the superior vena cava 142 when the occluder 602 is expanded.

As described with respect to FIGS. 3A-3D, occluding flow to the superior vena cava 142 can direct the blood that has been diverted from the right pulmonary artery 140 into the accessory hemiazygos vein 156, the hemiazygos vein 158, the internal thoracic veins 152, or other vessels including the intercostal veins, the internal mammary veins, and others. Such backflow may create a thoracic tank in the chest vessels, such as intercostals, the accessory hemiazygos vein 156, the hemiazygos vein 158, and others as discussed above.

FIG. 8A illustrates an example of the occluder system 600 with a collapsed occluder 602 of FIG. 6I in a venous system. FIG. 8B illustrates an example of the occluder system 600 with an expanded occluder 602 of FIG. 6J in a venous system.

The lead 608 can be positioned in the superior vena cava 142, the right brachiocephalic vein 118, and/or the right subclavian vein 114. In another embodiment, the lead can be positioned in the right internal jugular vein 110, the left brachiocephalic vein 120, the left internal jugular vein 112, or the left subclavian vein 116. The lead 608 can be connected to a control unit 450 as described in FIG. 4. In some embodiments, the occluder system 600 can lack a lead. In these embodiments, the control unit 450 can be connected directly to the drive motor 606.

As described above, one or more pressure sensors may be located along the occluder system 600. Various configurations are described below which are interchangeable or combinable. FIG. 9A illustrates an example of the occluder system 600 in a venous system of FIG. 8A with a sensor 914a proximal to the occluder 602 in the azygos vein 150 and a sensor 914b distal to the occluder 602 in the azygos vein 150. Sensor 914a can measure a pressure in the superior vena cava 142, or a central venous pressure. Sensor 914b can measure a pressure in the azygos vein 150. FIG. 9B illustrates an example of the occluder system 600 in a venous system of FIG. 8A with a sensor 914b distal to the occluder 602 in the azygos vein 150. Sensor 914b can measure a pressure in the azygos vein 150. FIG. 9C illustrates an example of the occluder system 600 in a venous system of FIG. 8A with a sensor 914c on the drive motor 606 in the superior vena cava 142. Sensor 914c can measure a pressure in the superior vena cava 142, or a central venous pressure. FIG. 9D illustrates an example of the occluder system 600 in a venous system of FIG. 8A with a sensor 914d on the occluder 602 in the azygos vein 150. Sensor 914d can measure a pressure in the azygos vein 150. FIG. 9E illustrates an example of the occluder system 600 in a venous system of FIG. 8A with a sensor 914e on a proximal end of the lead 608 in the right subclavian vein 114. Sensor 914e can measure a pressure in the right subclavian vein 114. FIG. 9F illustrates an example of the occluder system 600 in a venous system of FIG. 8A with a sensor 914f on a distal end of the lead 608 in the superior vena cava 142 or the right brachiocephalic vein 118. Sensor 914e can measure a pressure in the superior vena cava 142 or the right brachiocephalic vein 118. FIG. 9G illustrates an example of the occluder system 600 in a venous system of FIG. 8A with a sensor 914a proximal to the occluder 602 in the azygos vein 150. Sensor 914a can measure a pressure in the superior vena cava 142, or a central venous pressure.

The sensors 914a,b,c,d,e,f can be pressure sensors configured to measure pressure in a vessel. The control unit 450 can receive feedback from the sensors 914a,b,c,d,e,f as described in FIG. 4. The control unit 450 can control expansion of the occluder 602 based on the measurements of sensors 914a,b,c,d,e,f.

As shown in FIG. 9A, a first sensor 914a can measure pressure in the superior vena cava 142, for example near the azygos vein 150, and a second sensor 914b can measure pressure in the azygos vein 150, for example proximal to an outlet of a shunt 200. The sensor 914b can be positioned between the occluder 602 and the anchoring element 610. The control unit 450 (FIG. 4) can control expansion of the occluder 602 based on a pressure differential determined by the measurements of sensor 914a and sensor 914b. The occluder 602 can expand when the pressure differential is below a threshold and collapse when the pressure differential is above a threshold. As described with respect to FIG. 4, the control unit 450 can control expansion of the occluder 602 based on central venous pressure. In some embodiments, the control unit 450 can control expansion of the occluder 602 based on pressure in the azygos vein 150, right subclavian vein 114, right brachiocephalic vein 118, and/or right pulmonary artery 140. The threshold for controlling the occluder 602 based on a pressure measurement in any configuration can be similar to the thresholds described with respect FIG. 4 or a threshold specific to the location of measurement.

FIG. 10A shows a perspective view of an example of an occluder system 600 with the occluder 602 expanded. FIG. 10B shows a perspective view of the example of the expanded occluder 602 of FIG. 10A. FIG. 10C shows a side view of the example of the expanded occluder 602 of FIG. 10A. FIG. 10D shows a perspective view of the example of the collapsed occluder 602 of FIG. 10A. FIG. 10E shows a cross-sectional front view of the example of the collapsed occluder 602 of FIG. 10A. FIG. 10F shows a cross-sectional front view of the example of the expanded occluder 602 of FIG. 10A. FIG. 10G shows a cross-sectional view of the example of the expanded occluder 602 of FIG. 10A with the drive line 604 and shaft 1028 removed for clarity.

The occluder 602 can include an expandable cage 1020 attached to a proximal cap 1026 and a distal cap 1024. An elastic sheath 1022 can cover the expandable cage 1020. A shaft 1028 can extend through the expandable cage 1020. An inner distal cap 1030 can be inside the distal cap 1024 and a drive line connector 1032 can be inside the proximal cap 1026.

As shown in FIG. 10A, the occluder system 600 can include an occluder 602 at a distal portion of the occluder system 600. The occluder 602 can be positioned in a vessel, for example chronically implanted. The occluder 602 can expand (FIGS. 10B-10C) and collapse (FIG. 10D) to selectively occlude blood flow through the vessel. For example, the occluder 602 can be positioned in the azygos vein 150 to at least partially occlude blood flow through from the azygos vein 150 to the superior vena cava 142. The occluder 602 can include an expandable cage 1020 and/or an elastic sheath 1022. For example, the elastic sheath 1022 can cover and inner and/or outer surface of the expandable cage 1020. In some implementations, the elastic sheath 1022 can encapsulate the expandable cage 1020.

The expandable cage 1020 can extend between a proximal cap 1026 and a distal cap 1024. The expandable cage 1020 can be a wire form or a braid, for example made of nitinol. The expandable cage 1020 can be interlocking wires forming a diamond-like pattern.

The elastic sheath 1022 can be a smooth expandable material. The elastic sheath 1022 can minimize creasing, folding, and potential areas for thrombosis initiation. The occluder 602 can be covered in a thrombus reducing pharmaceutical, for example heparin. In some embodiments, the occluder 602 can partially expand or collapse based on pressure measurements or user input.

As shown in FIG. 10B-10C, the occluder system 600 can include a shaft 1028 extending through the occluder 602. The shaft may be a tubular body or may include a sealed distal end. The shaft 1028 can extend through the drive line 604 from a drive motor 606 toward a distal end of the occluder 602. The shaft 1028 can extend proximally past the proximal cap 1026. The shaft 1028 can extend distally past the distal cap 1024 when the distal cap 1024 is moved toward the proximal cap 1026, for example when the occluder 602 is expanded. The shaft 1028 can align with a distal edge of the distal cap 1024 when the distal cap 1024 is moved away from the proximal cap 1026, for example when the occluder 602 is collapsed. The distal end of the shaft 1028 can be coupled with an anchoring element 610 as described with respect to FIGS. 6K and 6L.

The proximal cap 1026 can move along the shaft 1028 of the occluder 602 toward the distal cap 1024 to expand the expandable cage 1020. The proximal cap 1026 can move along the shaft 1028 of the occluder 602 away from the distal cap 1024 to collapse the expandable cage 1020. In other configurations, the distal cap 1024 may move relative to the proximal cap 1026.

The proximal cap 1026 can be coupled, for example welded, to the drive line connector 1032. The drive line connector 1032 can be coupled, for example welded, to the drive line 604. In some embodiments, the drive line 604 can be coupled directly to the proximal cap 1026. In some embodiments, the proximal cap 1024 can move toward and away from the distal cap 1026 to expand and collapse the expandable cage 1020. When collapsed, the expandable cage 1020 can be positioned radially inward from the proximal cap 1026 and the distal cap 1024. When expanded, the expandable cage can expand radially outward relative to the proximal cap 1026 and the distal cap 1024. The expandable cage 1020 can be generally straight or tubular when collapsed and curve radially outward when expanded.

As seen in FIG. 10F, the ends of the wires that make up the expandable cage 1020 can align in circular arrangements radially outside the shaft 1028. Each of the proximal cap 1026 and the distal cap 1024 can include an outer ring and an inner ring positioned within the outer ring. As seen in FIG. 10G, the ends of the wires that make up the expandable cage 1020 can be positioned radially between the inner ring and the respective outer ring.

As shown in FIG. 10G, the drive line connector 1032 and the inner distal cap 1030 can be inside the proximal cap 1026 and the distal cap 1024. The drive line connector 1032 and the inner distal cap 1030 can house sensors, for example pressure sensors as described in FIGS. 9A, 9B, and 9G.

The elastic sheath 1022 can be attached to a distal portion of the shaft 1028 and/or the proximal and distal caps 1026, 1024. The elastic sheath 1022 can stretch as the expandable cage 1020 expands and contract when the expandable cage 1020 collapses.

The occluder 602 can connect to a drive motor 606 by the drive line 604. The drive motor 606 can be an in-line motor or a linear actuator motor. The drive motor 606 can cause the drive line 604 to expand and collapse the occluder 602. In some implementations, the drive motor 606 can be positioned in the superior vena cava. The drive line 604 can be positioned in the azygos vein and/or the superior vena cava.

The drive motor 606 can have an actuation length, or a length that it can push and pull the drive line 604 to expand and collapse the occluder 602. The drive motor 606 can have an actuation length of approximately 10 mm to fully open or close the occluder. In some embodiments, the drive motor 606 can have an actuation length of approximately 5-15 mm to fully open or close the occluder. In some embodiments, the drive motor 606 can have an actuation length of approximately 1-20 mm to fully open or close the occluder. The actuation length can be a moveable length of the drive line 604.

The drive motor 606 can be controlled by the controller 450, as described in FIG. 4. The drive motor 606 can be directly connected to the controller 450, as the lead 608 can be connected to the controller connection wire 452. In some embodiments, the occluder system 600 can be leadless. The drive motor 606 can connect directly to the controller 450, either by a wire or wirelessly. The drive motor 606 can be hermetically sealed to protect the motor from liquid and biological material.

The system described herein can be used to divert blood from the arterial system to the venous system. This can include diverting blood from the pulmonary artery to the azygos vein. The diversion of blood from an artery to a vein may be passive. The diversion of blood may be augmented through the use of an occluder positioned in the vein. The occluder may be positioned between an end of the shunt and an ostium from the vein to another vessel where blood flows antegrade toward the ostium. In some implementations, the artery and the vein may be non-parallel or generally extending in different directions relative to a superior-inferior direction.

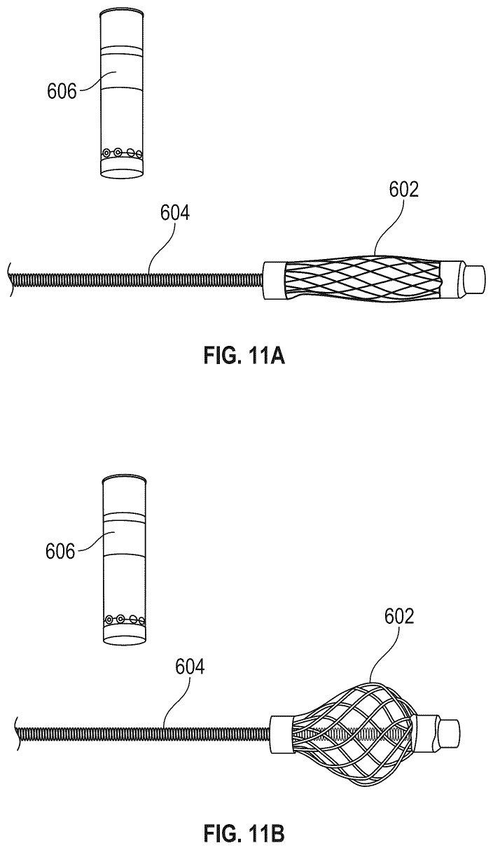

FIG. 11A illustrates an example of a collapsed occluder 602 and a drive line 604 detached from a drive motor 606. FIG. 11B illustrates an example of an expanded occluder 602 and a drive line 604 detached from a drive motor 606.

The collapsed occluder 602 can have a length of approximately 2 cm. In some embodiments, the collapsed occluder 602 can have a length of approximately 1-3 cm. In some embodiments, the collapsed occluder 602 can have a length of approximately 0.5-5 cm. The collapsed occluder 602 can have a width of approximately 0.25 cm. In some embodiments, the collapsed occluder 602 can have a width of approximately 0.1-0.5 cm. In some embodiments, the collapsed occluder 602 can have a width of approximately 0.01-1 cm.

The expanded occluder 602 can have a length of approximately 1.75 cm. In some embodiments, the expanded occluder 602 can have a length of approximately 1-3 cm. In some embodiments, the expanded occluder 602 can have a length of approximately 0.5-5 cm. The expanded occluder 602 can have a width of approximately 1 cm. In some embodiments, the expanded occluder 602 can have a width of approximately 0.5-2 cm. In some embodiments, the expanded occluder 602 can have a width of approximately 0.1-2.5 cm. In some embodiments, the expanded occluder 602 can have a width that corresponds to a width of the azygos vein 150, for example 0.9 cm.

The drive motor 606 can attach to a proximal end of the drive line 604. The proximal cap 1026 of the occluder 602 can be movable by the drive line 604. The drive motor 606 can have a length of approximately 1.75 cm. In some embodiments, the drive motor 606 can have a length of approximately 1-2 cm. In some embodiments, the drive motor 606 can have a length of approximately 0.5-4 cm. The drive motor 606 can have a width of approximately 0.25 cm. In some embodiments, the drive motor 606 can have a width of approximately 0.1-0.5 cm. In some embodiments, the drive motor 606 can have a width of approximately 0.01-1 cm.

FIG. 12 illustrates an example of a shunt 200 connecting an azygos vein 150 to a right pulmonary artery 140.

The shunt 200 can include a first flared end to seal against a puncture in the right pulmonary artery 140 and a second flared end to seal against a puncture in the azygos vein 150. A body portion of the shunt can extend between the flared ends. The body portion can be positioned between the artery and the vein. The shunt 200 can be made of a Nitinol braid or laser cut hypotube.

The implant or shunt 200 can include an expandable body 210 having lumen 213 between two ends. In some implementations, a first end of the shunt is positioned in the pulmonary artery, a second end of the shunt is positioned in the azygos vein, and a middle portion of the shunt is positioned at a connection between the pulmonary artery and the azygos vein. The expandable body 210 can be configured to collapse for delivery into the patient and expand into engagement with an inner wall of one or more vessels of the patient once implanted, with the expanded configuration shown. For example, a first or upstream portion of the shunt 200 may radially expand along and engage with a length of the pulmonary artery 140 upstream of the connection between the pulmonary artery 140 and the azygos vein 150, with respect to a direction of blood flow in the pulmonary artery. A second or downstream portion of the shunt 200 may radially expand along and engage with a length of the azygos vein 150 upstream of the connection between the right pulmonary artery 140 and the azygos vein 150, with respect to a direction of blood flow in the azygos vein. Once implanted, blood flowing through the vessel(s) in which the implant 200 is implanted can flow through the lumen 213.

The shunt 200 may include an inner body located within an outer body. In some implementations, an implant such as shunt 200 may include an expandable body with an outer body of an expandable membrane or fabric layer at least partially covering an inner body of a metallic frame comprising one or more struts as discussed above. In some implementations, shunt 200 comprising an inner body made of an expandable tubular frame (e.g., nitinol) that may comprise struts. The struts may be covered with an outer body, such as a membrane or fabric of biocompatible material, for example PTFE or ePTFE. In some implementations, the expandable body can include an expandable membrane or fabric inner body surrounded or partially surrounded by a tubular outer body of an expandable stent-like metallic frame as described above.

In some implementations, the shunt 200 has a length sufficient to connect the pulmonary artery 140 to the azygos vein 150. In some implementations, each end of the shunt 200 may include a flare, barbs, hooks, or other anchor structures to help secure the end in a respective vessel. In some implementations, the shunt 200 can have two collapsible flared ends on each side of the shunt 200. In some implementations, the two collapsible flared ends can engage both an inner and outer vessel wall to keep the shunt 200 in place. In some implementations, the two collapsible flared ends can expand radially from the shunt 200 when the shunt is between two vessels and grip the outer and inner wall of each vessel. For example, in some implementations, the two collapsible flared ends of the shunt 200 can engage an outer and inner wall of an azygos vein 150 on one end and an outer and inner wall of a right pulmonary artery 140 on the other end. The shunt 200 can be similar to the shunt 1372 described with respect to FIGS. 13A-13E and 14A-14B.

In some implementations, shunt 200 may be tubular and have a sufficient length to radially expand against and extend along a length of the pulmonary artery 140 to secure an upstream or entrance end of the shunt 200 and direct blood from the pulmonary artery 140 into the shunt 200. The length may also be sufficient to radially expand against and extend along a length of the azygos vein 150, for example to secure a downstream or exit end of the shunt 200 and direct blood into the azygos vein 150. In some implementations, the downstream or exit end of shunt 200 is configured to direct blood upstream in the azygos vein 150, toward the thoracic vessels and splanchnic cavity described above. In some implementations, the downstream or exit end of shunt 200 is configured to direct blood downstream in the azygos vein 150, toward the superior vena cava 142.

The shunt 200 can be configured to collapse and expand. Additionally, the outer body and the inner body can be configured to collapse and expand together. For example, in some implementations, the outer body 215 and inner body 225 are configured to be implanted together and expand together within the target vessels. In some implementations, the outer body and inner body are configured to be implanted separately or serially. For example, outer body may be implanted and expanded first, then inner body may be implanted and expanded within the outer body 215.

In some implementations, the implant may be a short shunt 200 configured to provide a connection between two vessels (e.g., between the pulmonary artery 140 and the azygos vein 150). This shunt 200 may preferably be positioned between the pulmonary artery 140 and the azygos vein 150 without extending substantially into the lumen of either vessel. As illustrated in side view FIG. 3C, the shunt 200 may be implanted to create a flow pathway between a branch of the pulmonary artery 140 and the azygos vein 150. This location allows blood flow 304 to continue along forward or antegrade direction 304A within the branch of the pulmonary artery 140 toward the lung, as normal, and a portion of the blood flow 304 to be diverted through the shunt 200 along direction 304B into the azygos vein 150. As illustrated in top view FIG. 3D, blood flow 304 exits the shunt 200 into the azygos vein 150 and may provide backflow along reverse or retrograde direction 304C. Such backflow may create a thoracic tank in the chest vessels, such as intercostals, the accessory hemiazygos vein 156, the hemiazygos vein 158, and others as discussed above. A portion of the diverted blood may also enter the superior vena cava 142. As described further below, in some implementations the shunt 200 may comprise a controllable valve. Also as described further below, in some implementations blood flow from the azygos vein 150 to the superior vena cava 142 may be separately restricted or occluded, such as by positioning an additional flow restricting implant within the azygos vein 150 downstream of the shunt 200.

In some embodiments, the shunt 200 can include a check valve. A check valve may selectively direct blood from the pulmonary artery 140 to the azygos vein 150 and/or the superior vena cava 142. For example, a check valve may be located along the length of the shunt 200 to selectively allow blood from the pulmonary artery 140 in a forward direction in the azygos vein 150 into the superior vena cava 142. The check valve may be located on a portion of the shunt 200 that is downstream from the connection between the pulmonary artery 140 and the azygos vein 150. In some implementations, check valve may be a pressure valve designed to open after a desired amount of blood has been diverted to the thoracic tank. For example, in some implementations, the check valve can be a leaflet or pop valve designed to open under a predetermined pressure. In some implementations, the check valve may be an aperture or iris designed to permit a predetermined volume of blood from the pulmonary artery 140 into the azygos vein 150.

Some implementations of the check valve may be located within the azygos vein 150 after implantation of the shunt 200. In some implementations, the check valve may be located on a side of the shunt 200 closer to the superior vena cava 142. In some implementations, the check valve may be a one-way valve to control the direction of blood flow. In some implementations, the check valve may be located in the azygos vein 150 closer to the thoracic tank, for example at the end of the shunt 200 located within the azygos vein 150 upstream of the connection between the pulmonary artery 140 and the azygos vein 150. In some implementations, the check valve may be located within the pulmonary artery 140 to control an amount of blood diverted from the pulmonary artery 140 through the shunt 200 and into the azygos vein 150. In some implementations, the check valve may be configured to be positioned within the shunt 200 between the pulmonary artery 140 and the azygos vein 150 after implantation. In some embodiments, multiple check valves (e.g., two valves or three valves) may be positioned along the shunt 200 at any of the locations described above.

FIG. 13A illustrates an example of a shunt 1372 crimped in a shunt sheath 1370. FIG. 13B illustrates an inner arterial arm 1374a of the shunt 1372 engaging an inner wall of a right pulmonary artery 140. FIG. 13C illustrates the outer arterial arm 1376a engaging an outer wall of the right pulmonary artery 140. FIG. 13D illustrates the outer venous arm 1376b engaging an outer wall of an azygos vein 150. FIG. 13E illustrates the inner venous arm 1374b engaging an inner wall of the azygos vein 150. The shunt 1372 can be similar to the shunt 200 of FIG. 12. The shunt 1372 can be positioned using a guidewire or guide sheath. In some embodiments, the shunt sheath 1370 can be a guide sheath.

As shown in FIG. 13A, the shunt sheath 1370 can keep the shunt 1372 collapsed. The shunt 1372 can be biased such that the inner arterial arm 1374a, outer arterial arm 1376a, inner venous arm 1374b, and outer venous arm 1376b expand radially outward when the shunt sheath 1372 is removed.

As shown in FIG. 13B, the shunt sheath 1370 can be partially removed, or slid off, the shunt 1372, when positioned at least partially in an artery. The inner arterial arm 1374a of the shunt 1372 can expand radially outward to engage the inner wall of the artery, for example the right pulmonary artery 140.

As shown in FIG. 13C, the shunt sheath 1370 can be further removed, or slid off, the shunt 1372. The outer arterial arm 1376a of the shunt 1372 can expand radially outward to engage the outer wall of the artery, for example the right pulmonary artery 140.

As shown in FIG. 13D, the shunt sheath 1370 can be partially removed, or slid off, the shunt 1372, when positioned at least partially in a vein. The inner venous arm 1374b of the shunt 1372 can expand radially outward to engage the inner wall of the vein, for example the azygos vein 150.

As shown in FIG. 13E, the shunt sheath 1370 can be further removed, or slid off, the shunt 1372. The outer arterial arm 1376b of the shunt 1372 can expand radially outward to engage the outer wall of the vein, for example the azygos vein 150. The shunt sheath 1370 can be removed from the vein, for example using a guidewire.

The inner arterial arm 1374a, outer arterial arm 1376a, inner venous arm 1374b, and outer venous arm 1376b can contact a vessel wall around a connection point, for example 360 degrees around the connection point to form a seal with the respective vessel walls. In some embodiments, the arms can include one or more portions for contacting the vessel wall less than 360 degrees around the connection point, for example at least about 90 degrees and/or less than or equal to about 360 degrees or at least about 180 degrees and less than or equal to about 360 degrees. In some embodiments, the arms can contact the vessel wall at one to ten points around the connection point.

FIG. 14A illustrates an example of a shunt 1372. FIG. 14B illustrates the example of the shunt 1372 of FIG. 14A secured to a right pulmonary artery 140.

The inner arterial arm 1374a, outer arterial arm 1376a, inner venous arm 1374b, and outer venous arm 1376b can be circular or ovular. The inner arterial arm 1374a, outer arterial arm 1376a, inner venous arm 1374b, and outer venous arm 1376b can be curved to engage an optimal surface area of the vessel walls. The inner arms 1374a,b and outer arms 1376a,b can be biased toward each other to grip the vessel wall. The inner arms 1374a,b and outer arms 1376a,b can pinch the vessel walls with a force. Advantageously, this force can prevent endoleaks.

The shunt 1372 can be covered in ePTFE or PET. The shunt 1372 and/or the arms can be Nitinol. The shunt 1372 can have ePTFE or PET between the inner and outer arms. The outer arms 1376a,b can be smaller than the inner arms 1374a,b. The arms can be flanges, for example saddle shaped flanges to accommodate a vessel diameter. Advantageously, this can allow for a better seal and better vessel approximation.

In some implementations, a method for treating heart failure includes diverting blood from a pulmonary artery 140 to the azygos vein 150. An implanted device, such as shunt 200 or an occluder system 600, may be used to divert the blood. In some implementations, the diverting of blood is sufficient to decongest lungs of the patient and reduce a left ventricular end diastolic pressure (LVEDP) and/or to reduce pulmonary artery pressure to relieve pulmonary hypertension and consequently reduce a workload of a right ventricle of the patient. In some implementations, the diverting of blood is sufficient to mimic a splanchnic vascular capacitance and redistribute blood into a splanchnic compartment of the patient. In some implementations, the diverting of blood is sufficient to cause dilation and/or increased pressure within intercostal veins of the patient.

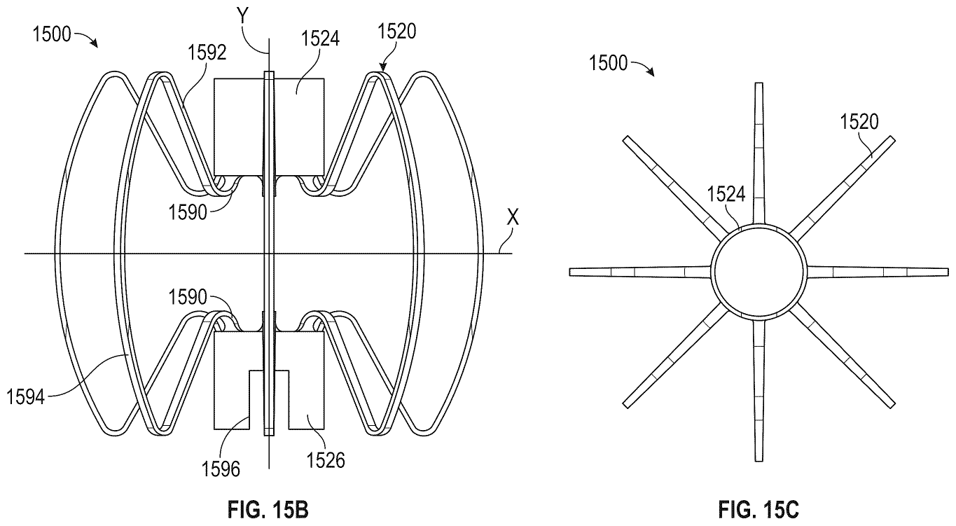

FIG. 15A shows a perspective view of another occluder 1500. FIG. 15B shows a side view of the occluder 1500 of FIG. 15A. FIG. 15C shows a top view of the occluder 1500 of FIG. 15A.

The occluder 1500 can include a distal cap 1524 and a proximal cap 1526 connected by a plurality of members 1520 that make up an expandable cage. The members 1520 can be circumferentially disposed about a central axis of the occluder 1500.

As shown in FIG. 15B, the members 1520 can be fixed to the inner edges of the distal cap 1524 and proximal cap 1526. Each member 1520 can have a first end positioned on a first cap and a second end positioned on a second cap. The members 1520 can be fixed to the bottom edge of the distal cap 1524 along the vertical axis Y. The members 1520 can be fixed to the upper edge of the proximal cap 1526 along the vertical axis Y. The members 1520 can extend radially outward from the distal cap 1524 and proximal cap 1526. A first portion 1590 of each member 1520 can extend from the distal cap 1524 and proximal cap 1526 toward the central horizontal axis X. A second portion 1592 of each member 1520 can extend from the first portion 1590 of the member 1520 away from the central horizontal axis X. The second portion 1592 of each member 1520 can be straight. A third portion 1594 of each member 1520 can extend between the second portions 1592 extending from each cap 1524, 1526. The third portion 1594 of each member 1520 can be curved radially outward. The portions 1590, 1592, 1594 of each member 1520 can be connected by curved portions. The members 1520 can be shaped as wings. The members 1520 can be malleable or flexible. The members 1520 can be biased to expand radially outward.

In some implementations, the occluder can include 8 members 1520. In some implementations, the occluder can include 4 to 12 members 1520. In some implementations, the occluder can include 1 to 20 members 1520.

The occluder 1500 can be a twist occluder, or an occluder that expands and collapses when subjected to rotational forces. The members 1520 can collapse inward when subjected to a first rotational force. The members 1520 can expand outward when subjected to a second rotational force, for example a rotational force opposite the first rotational force. In some implementations, the occluder 1500 can be expanded and collapsed by rotating the distal cap 1524 relative to the proximal cap 1526. In other implementations, the occluder 1500 can be expanded and collapsed by rotating the proximal cap 1526 relative to the distal cap 1524.

At least one of the proximal cap 1526 or the distal cap 1524 can include a slot 1596. The slot 1596 can allow the cap 1524, 1526 to be easily rotated with respect to the other cap. A cap 1524, 1526 with a slot 1596 can be easily rotated by implementing a rotational force on an inner edge of the slot 1596. A drive line can be controlled by a drive motor to individually rotate one cap of the occluder 1500 with respect to the other. For example, the drive line can include a portion configured to implement a rotational force on an inner edge of the slot 1596 and a portion configured to keep the cap without the slot 1596 in place.

In some examples, the occluder 1500 can include an elastic sheath covering the members 1520. The elastic sheath can be a smooth expandable material. The elastic sheath can minimize creasing, folding, and potential areas for thrombosis initiation. As described herein, the occluder 1500 can expand and contract based on pressure measurements and/or user input.

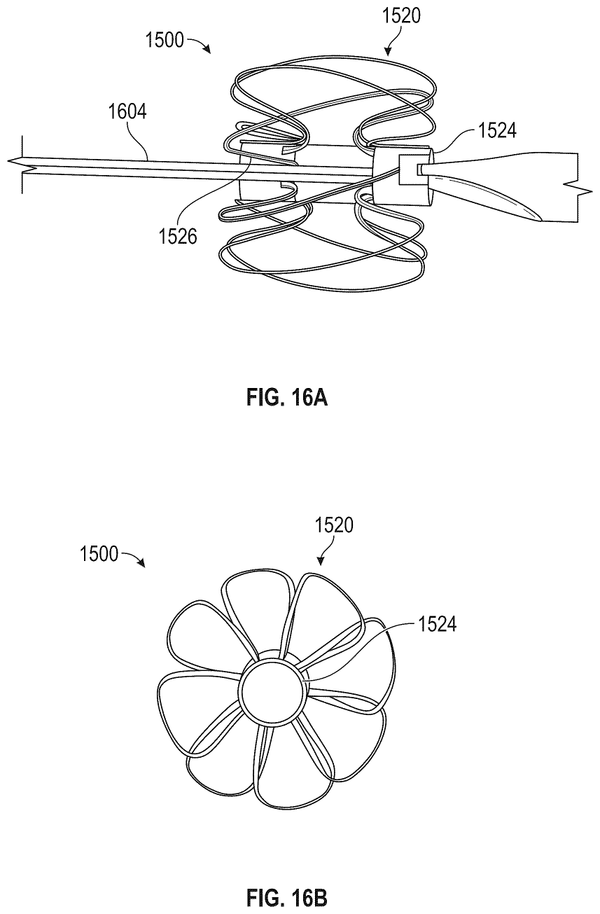

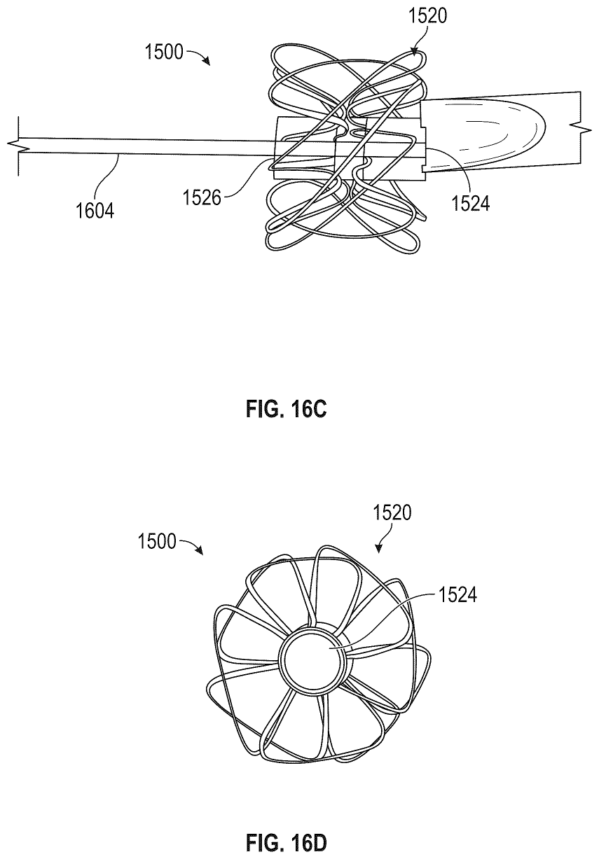

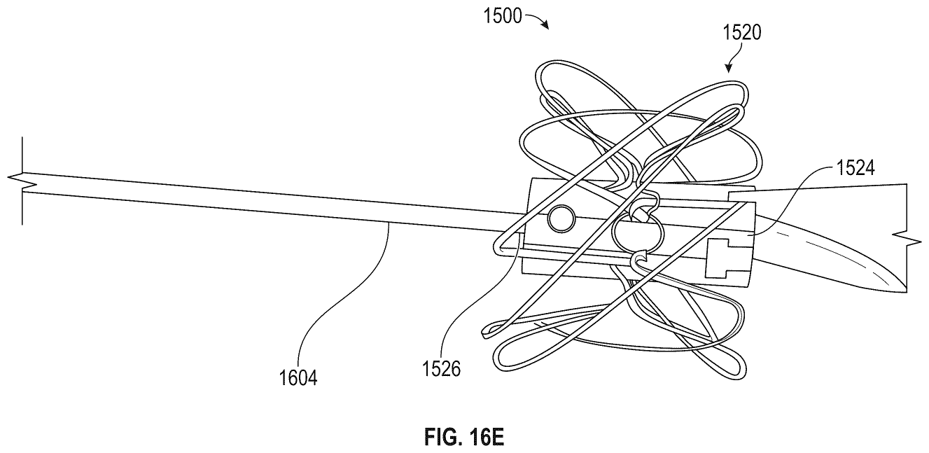

FIG. 16A shows a side view of the occluder 1500 of FIG. 15A connected to a drive line 1604, the occluder 1500 in a partially collapsed state. FIG. 16B shows a front view of the occluder 1500 of FIG. 15A in the partially collapsed state. FIG. 16C shows a side view of the occluder 1500 of FIG. 15A connected to a drive line 1604, the occluder 1500 in a further collapsed state. FIG. 16D shows a front view of the example of the occluder 1500 of FIG. 15A in the further collapsed state. FIG. 16E shows a side view of the occluder 1500 of FIG. 15A connected to a drive line 1604, the occluder 1500 in a further collapsed state.

The occluder 1500 can be similar to the occluder described with respect to FIGS. 15A-15C. The occluder 1500 can be positioned on an end of a drive line 1604. The drive line 1604 can be configured to rotate one of the proximal cap 1526 or the distal cap 1524 of the occluder 1500 with respect to the other of the proximal cap 1526 or the distal cap 1524. Rotating the first cap of the occluder 1500 with respect to the second cap can cause expansion and/or contraction of the occluder 1500.

As shown in FIGS. 16A and 16B, the proximal and distal end of each member 1520 of the occluder 1500 can be radially offset when partially collapsed. As a non-limiting example, the first cap in the shown embodiment is rotated approximately 50 degrees with respect to the second cap. In the partially collapsed state, the members 1520 can be positioned at an angle relative to the longitudinal axis because the proximal and distal ends of the members 1520 are not radially aligned. As the occluder is collapsed by rotation of a cap 1524, 1526 with respect to the other, the radius to which the members 1520 extend outward can decrease.

As shown in FIGS. 16C and 16D, the proximal and distal end of each member 1520 of the occluder 1500 can be further radially offset when further collapsed. As a non-limiting example, the first cap in the shown embodiment is rotated approximately 100 degrees with respect to the second cap. In the further collapsed state, the members 1520 can be at slanted angles because the proximal and distal ends of the members 1520 are not radially aligned. The end of each member 1520 on the cap being rotated can pass the initial radial position of the end of the adjacent member 1520. This can cause the members 1520 to overlap with one another as the occluder 1500 is collapsed. As the occluder is further collapsed by rotation of a cap 1524, 1526 with respect to the other, the radius to which the members 1520 extend can decrease further.

As shown in FIG. 16E, the proximal and distal end of each member 1520 of the occluder 1500 can be further radially offset when further collapsed. As a non-limiting example, the first cap in the shown embodiment is rotated approximately 150 degrees with respect to the second cap. As the occluder is further collapsed by rotation of a cap 1524, 1526 with respect to the other, the radius to which the members 1520 extend can decrease further. In some implementations, the occluder 1500 can alternate between expanded and collapsed states by rotating a cap with respect to the other cap by between 140 and 160 degrees of rotation. In some implementations, the occluder 1500 can alternate between expanded and collapsed states by rotating a cap with respect to the other cap by between 120 and 180 degrees of rotation. In some implementations, the occluder 1500 can alternate between expanded and collapsed states by rotating a cap with respect to the other cap by between 90 and 210 degrees of rotation. In some implementations, the occluder 1500 can alternate between expanded and collapsed states by rotating a cap with respect to the other cap by between 45 and 300 degrees of rotation.

The drive line 1604 can be rotated by a drive motor. The drive line 1604 can rotate a first cap of the occluder 1500 with respect to a second cap in a first direction to expand the occluder 1500. The drive line 1604 can rotate a first cap of the occluder 1500 with respect to a second cap in a second direction to contract the occluder 1500. The drive line 1604 can include a portion that keeps the second cap in a radial position while rotating the first cap.

In some examples, the occluder 1500 can include an elastic sheath covering the members 1520. The elastic sheath can be a smooth expandable material. The elastic sheath can minimize creasing, folding, and potential areas for thrombosis initiation. As described herein, the occluder 1500 can expand and contract based on pressure measurements and/or user input.

Some of the features or advantages encompassed by one or more of the above embodiments, or other aspects of the present application, include, but are not limited, to one or more of the following:
Increasing cardiac output by pressurizing or dilating veins, e.g., the intercostal veins;
Accommodating diverted blood in a venous capacitance system;
Diversion of blood is sufficient to decrease tension and stretch on a left ventricular wall which increases the tissue levels of vaso-constrictors like norepinephrine, Angiotensin II, and certain cytokines which have a deleterious effect on the heart and the vascular system, which result in exacerbation of heart failure;
Devices and methods for placing a shunt or creating a fistula between the right pulmonary artery and the azygos vein, to redirect blood through one or more pathways back into the right atrium;
Reducing stress on the lungs and reducing LVDP by diverting blood from the pulmonary artery to reduce the amount of blood that reaches the lungs;
A shunt and/or occluder to control the flow of blood between two body locations based on a feedback loop accounting for pressures taken from various locations in the body;
Mimicking a splanchnic vascular capacitance and redistributing blood into a venous system of the patient;
Diverting of blood to counteract vaso-constrictors released by the patient as a result of heart failure;
Placing the occluder system in the coronary sinus can be advantageous to treat chronic refractory angina by increasing blood pressure in the heart.

### Additional Embodiments

Clause 1. A system for treating heart failure of a patient, the system comprising: a shunt configured to direct blood flow from a pulmonary artery to an azygos vein; an expandable occluder configured to be positioned within the azygos vein to at least partially occlude blood flow through the azygos vein into a superior vena cava; and an implantable control unit configured to regulate expansion of the expandable occluder to cause blood flow from the pulmonary artery through the shunt to the azygos vein in a retrograde direction through the azygos vein.

Clause 2. The system of clause 1, wherein the shunt comprises a check valve.

Clause 3. The system of clause 1, wherein the shunt is configured to passively allow blood to flow from the pulmonary artery to the azygos vein.

Clause 4. The system of claim any one of the preceding clauses, wherein the shunt comprises a first flared end configured to seal against a puncture in a pulmonary artery and a second flared end configured to seal against a puncture in an azygos vein.

Clause 5. The system of claim any one of the preceding clauses, further comprising an anchoring element configured to anchor the expandable occluder within the azygos vein.

Clause 6. The system of clause 5, wherein the anchoring element comprises an expandable stent configured to engage inner walls of the azygos vein.

Clause 7. The system of clause 5 or 6, wherein the anchoring element is removably connectable to the expandable occluder.

Clause 8. The system of claim any one of the preceding clauses, further comprising a drive motor connectable to the expandable occluder, the drive motor configured to expand and collapse the expandable occluder.

Clause 9. The system of clause 7, wherein the drive motor is configured to be positioned in a superior vena cava.

Clause 10. The system of clause 8 or 9, further comprising a lead connectable to the drive motor and the implantable control unit.

Clause 11. The system of clause 10, wherein the lead is configured to be positioned in a right subclavian vein.

Clause 12. The system of any one of the preceding clauses, wherein the control unit is configured to be implanted subcutaneously.

Clause 13. The system of any one of the preceding clauses, further comprising one or more implantable pressure sensors, the implantable control unit configured to receive pressure measurements from the one or more implantable pressure sensors, the implantable control unit configured to control the expandable occluder based on the pressure measurements.

Clause 14. The system of clause 13, wherein the one or more implantable pressure sensors are configured to be positioned in the superior vena cava.

Clause 15. The system of clause 13 or 14, wherein the one or more implantable pressure sensors are positioned between in the azygos vein.

Clause 16. The system of any one of the preceding clauses, wherein the implantable control unit comprises an accelerometer and/or an electrocardiogram.

Clause 17. A method of treating heart failure of a patient, the method comprising: positioning a shunt to direct blood flow from a pulmonary artery to an azygos vein; positioning an expandable occluder within the azygos vein to at least partially occlude blood flow through the azygos vein into a superior vena cava; and expanding the expandable occluder based on feedback received from one or more pressure sensors to at least partially occlude blood flow through the azygos vein such that blood flows from the pulmonary artery through the shunt to the azygos vein in a retrograde direction through the azygos vein.

Clause 18. The method of clause 17, wherein receiving the feedback from the one or more pressure sensors comprises measuring a first pressure measurement in the azygos vein using a first implantable sensor positioned in the azygos vein and a second pressure measurement in a superior vena cava.

Clause 19. The method of clause 17, further comprising determining a central venous pressure based on the feedback from the one or more pressure sensors.

Clause 20. The method of clause 17, wherein expanding the expandable occluder based on feedback received from the one or more pressure sensors comprises expanding the expandable occluder when central venous pressure exceeds a threshold pressure value.

Clause 21. The method of clause 20, wherein the threshold pressure value is 15 mmHg.

Clause 22. The method of clause 17, further comprising collapsing the expandable occluder when central venous pressure falls below a threshold pressure value.

Clause 23. The method of clause 22, wherein the threshold pressure value is 5 mmHg.

Clause 24. The method of clause 17, further comprising: collecting, by an accelerometer, one or more activity measurements; and expanding the expandable occluder based at least in part on the one or more activity measurements.

Clause 25. The method of clause 17, further comprising: receiving, by an electrocardiogram, one or more electrical activity measurements; and expanding the expandable occluder based at least in part on the one or more electrical activity measurements.

Clause 26. The method of clause 17, wherein expanding the expandable occluder comprises moving, by a drive motor, a first end of the expandable occluder closer to a second end of the expandable occluder and moving, by the drive motor, the first end of the expandable occluder away from the second end of the expandable occluder.

Clause 27. The method of clause 17, further comprising transmitting the feedback received from the one or more pressure sensors to an external device.

Clause 28. A system for treating heart failure of a patient, the system comprising: a shunt configured to direct blood flow from an artery to a vein; an occluder configured to be positioned away from the shunt and at least partially occlude blood flow through the vein; and an implantable control unit configured to regulate expansion of the occluder.

Clause 29. The system of clause 28, wherein the shunt is configured to be positioned between a pulmonary artery and an azygos vein.

Clause 30. The system of any one of clauses 28-29, wherein the occluder is configured to at least partially occlude blood flow from an azygos vein into a superior vena cava.

Clause 31. The system of any one of clauses 28-30, wherein the shunt comprises a check valve.

Clause 32. The system of any one of clauses 28-31, wherein the shunt comprises a first flared end configured to seal against a puncture in a pulmonary artery and a second flared end configured to seal against a puncture in an azygos vein.

Clause 33. The system of any one of clauses 28-32, further comprising an anchoring element configured to anchor the occluder.

Clause 34. The system of clause 33, wherein the anchoring element comprises a stent.

Clause 35. The system of clause 33, wherein the anchoring element is removably connectable to the occluder.

Clause 36. The system of clause 33, wherein the anchoring element is connectable to the occluder by a plurality of anchoring wires.

Clause 37. The system of any one of clauses 28-33, further comprising a drive motor configured to expand and collapse the occluder.

Clause 38. The system of clause 37, wherein the drive motor is an in-line motor.

Clause 39. The system of clause 37, further comprising a lead connectable to the drive motor, the lead connectable to the implantable control unit.

Clause 40. The system of any one of clauses 28-33, further comprising one or more implantable pressure sensors, the implantable control unit configured to receive pressure measurements from the one or more implantable pressure sensors.

Clause 41. The system of clause 40, wherein the one or more implantable pressure sensors are positioned on the occluder.

Clause 42. The system of clause 40, wherein the one or more implantable pressure sensors are positioned between the occluder and an anchoring element.

Clause 43. The system of clause 40, wherein the implantable control unit is configured to automatically control the occluder based on the pressure measurements.

Clause 44. The system of clause 37, wherein the drive motor is configured to be positioned in a superior vena cava.

Clause 45. The system of clause 39, wherein the lead is configured to be positioned in a right subclavian vein.

Clause 46. The system of any one of clauses 28-33, wherein the shunt is configured to passively allow blood to flow from the artery to the vein.

Clause 47. The system of clause 40, wherein the implantable control unit is configured to expand the occluder when the pressure measurement is above an upper threshold.

Clause 48. The system of clause 47, wherein the upper threshold is 15 mmHg.

Clause 49. The system of clause 40, wherein the implantable control unit is configured to collapse the occluder when the pressure measurement is below a lower threshold.

Clause 50. The system of clause 49, wherein the lower threshold is 5 mmHg.

Clause 51. The system of any one of clauses 28-33, wherein the implantable control unit comprises a wireless charging coil.

Clause 52. The system of any one of clauses 28-33, wherein the implantable control unit comprises an accelerometer.

Clause 53. The system of any one of clauses 28-33, wherein the implantable control unit comprises an electrocardiogram.

Clause 54. The system of any one of clauses 28-33, wherein the implantable control unit comprises a wireless transmitter.

Clause 55. The system of any one of clauses 28-33, wherein the occluder comprises an expandable cage.

Clause 56. The system of clause 55, wherein the expandable cage comprises a Nitinol braid.

Clause 57. The system of clause 37, wherein the drive motor is configured to expand the occluder by moving a distal end of the occluder closer to a proximal end of the occluder and collapse the occluder by moving the distal end of the occluder away from the proximal end of the occluder.

Clause 58. The system of clause 37, wherein the drive motor is configured to expand the occluder by moving a proximal end of the occluder closer to a distal end of the occluder and collapse the occluder by moving the proximal end of the occluder away from the distal end of the occluder.

Clause 59. A chronically implantable occluder system comprising: an occluder comprising an expandable cage; an anchoring element positionable distal to the occluder, the anchoring element configured to anchor the occluder within a patient's vessel; and an implantable control unit configured to activate the occluder.

Clause 60. The occluder system of clause 59, wherein the anchoring element is removably attachable to the expandable cage.

Clause 61. The occluder system of any one of clauses 59-60, further comprising a drive motor connectable to the expandable cage, the drive motor configured to expand and collapse the expandable cage.

Clause 62. The occluder system of clause 61, wherein the drive motor is connectable to the expandable cage by a drive wire.

Clause 63. The occluder system of clause 61, further comprising a lead connectable to the drive motor, the lead connectable to the implantable control unit.

Clause 64. The occluder system of clause 63, wherein the lead is wirelessly connectable to the implantable control unit.

Clause 65. The occluder system of clause 61, wherein the drive motor is connectable to the implantable control unit.

Clause 66. The occluder system of clause 61, wherein the drive motor is configured to be positioned in a superior vena cava.

Clause 67. The occluder system of any one of clauses 59-61, wherein the expandable cage comprises a Nitinol braid.

Clause 68. The occluder system of any one of clauses 59-61, wherein the expandable cage is covered in a smooth expandable material.

Clause 69. The occluder system of any one of clauses 59-61, wherein the expandable cage is covered in a thrombus reducing pharmaceutical.

Clause 70. The occluder system of clause 61, wherein the drive motor is configured to expand the expandable cage by moving a distal end of the expandable cage closer to a proximal end of the expandable cage and collapse the expandable cage by moving the distal end of the expandable cage away from the proximal end of the expandable cage.

Clause 71. The occluder system of clause 61, wherein the drive motor is configured to expand the expandable cage by moving a proximal end of the expandable cage closer to a distal end of the expandable cage and collapse the expandable cage by moving the proximal end of the expandable cage away from the distal end of the expandable cage.

Clause 72. The occluder system of any one of clauses 59-61, wherein the anchoring element comprises a stent.

Clause 73. The occluder system of any one of clauses 59-61, wherein the anchoring element is connectable to the expandable cage by a plurality of anchoring wires.

Clause 74. The occluder system of any one of clauses 59-61, wherein the anchoring element is a J-tip.

Clause 75. The occluder system of any one of clauses 59-61, wherein the anchoring element does not obstruct collateral flow.

Clause 76. The occluder system of any one of clauses 59-61, wherein the implantable control unit comprises a wireless transmitter.

Clause 77. The occluder system of any one of clauses 59-61, wherein expanding the expandable cage causes the expandable cage to contact a wall of the vessel.

Clause 78. A shunt configured to be positioned between a pulmonary artery and an azygos vein, the shunt comprising: a body portion extending between a first flared end and a second flared end; the first flared end configured to be deployed within a pulmonary artery and anchor around a puncture in a pulmonary artery; and the second flared end configured to be deployed within an azygos vein and anchor around a puncture in an azygos vein.

Clause 79. The shunt of clause 78, further comprising a check valve.

Clause 80. The shunt of any one of clauses 78-79, wherein the body portion is configured to passively allow blood to flow from the pulmonary artery to the azygos vein.

Clause 81. The shunt of any one of clauses 78-80, wherein the body portion comprises a Nitinol braid.

Clause 82. The shunt of any one of clauses 78-81, wherein the body portion comprises a laser cut hypotube.

Clause 83. The shunt of any one of clauses 78-82, wherein the body portion is covered in ePTFE.

Clause 84. A method of treating heart failure of a patient, the method comprising: receiving, by a control unit implanted in a patient, one or more pressure measurements collected using one or more implantable sensors; and activating, by the control unit, an occluder implanted in an azygos vein of the patient based on the one or more pressure measurements, wherein activating the occluder causes blood to flow from a pulmonary artery to the azygos vein via a shunt implanted between a pulmonary artery and the azygos vein.

Clause 85. The method of clause 84, wherein collecting the one or more pressure measurements comprises measuring a first pressure measurement measured in the azygos vein using a first implantable sensor positioned at an outlet of a shunt and a second pressure measurement measured in a superior vena cava.

Clause 86. The method of any one of clauses 84-85, further comprising determining, by the control unit, a central venous pressure based on the one or more pressure measurements.

Clause 87. The method of any one of clauses 84-86, wherein activating the occluder comprises expanding the occluder when central venous pressure exceeds a threshold pressure value.

Clause 88. The method of clause 87, wherein the threshold pressure value is 15 mmHg.

Clause 89. The method of any one of clauses 84-87, further comprising deactivating the occluder when central venous pressure falls below a threshold pressure value.

Clause 90. The method of clause 89, wherein the threshold pressure value is 5 mmHg.

Clause 91. The method of any one of clauses 84-87, further comprising activating a drive motor via the control unit to expand or collapse the occluder.

Clause 92. The method of any one of clauses 84-87, further comprising transmitting, by the control unit, the one or more pressure measurements to a smart device.

Clause 93. The method of any one of clauses 84-87, further comprising charging, by a wireless charging coil, the control unit.

Clause 94. The method of any one of clauses 84-87, further comprising: collecting, by an accelerometer, one or more activity measurements; and activating, by the control unit, the occluder based at least in part on the one or more activity measurements.

Clause 95. The method of any one of clauses 84-87, further comprising: receiving, by an electrocardiogram, one or more electrical activity measurements; and activating, by the control unit, the occluder based at least in part on the one or more electrical activity measurements.

Clause 96. A method of implanting a system for treating heart failure, the method comprising: passing a guidewire between an artery and a vein; advancing a shunt over the guidewire; implanting a first end of the shunt in the artery and a second end of the shunt in the vein; advancing an occluder system over the guidewire; positioning an expandable element of the occluder system in the vein; anchoring the expandable element using an anchoring element positioned in the vein; and subcutaneously implanting a control unit configured to activate the expandable element.

Clause 97. The method of clause 96, wherein the artery is a pulmonary artery and the vein is an azygos vein.

Clause 98. The method of clause 97, further comprising positioning a drive line of the occluder system in a superior vena cava, the drive line extending between the expandable element and the control unit.

Clause 99. The method of clause 96, further comprising disconnecting the anchoring element from the expandable element.

Clause 100. A method of implanting a system for treating heart failure, the method comprising: passing a guidewire between a pulmonary artery and an azygos vein; advancing a shunt over the guidewire; and implanting a first end of the shunt in a pulmonary artery and a second end of the shunt in an azygos vein.

Clause 101. The method of clause 100, further comprising advancing the shunt from the pulmonary artery to the azygos vein.

Clause 102. The method of clause 100, further comprising advancing the shunt from the azygos vein to the pulmonary artery.

Clause 103. A method for controlling flow through a vessel in a patient, the method comprising: receiving, by a control unit implanted in a patient, one or more pressure measurements collected using one or more implantable sensors; transmitting, by the one or more implantable sensors, a central venous pressure to a control unit implanted in the patient, the control unit configured to activate a drive motor in response to the measured central venous pressure; and activating, by the control unit implanted in the patient, the drive motor to expand an expandable cage implanted in the vessel by moving one end of the expandable cage toward an opposite end of the expandable cage.

Clause 104. The method of clause 103, wherein activating the drive motor pulls a distal end of the expandable cage toward a proximal end of the expandable cage to expand the expandable cage.

Clause 105. The method of any one of clauses 103-104, wherein the control unit is configured to activate the drive motor to expand the expandable cage when the central venous pressure exceeds a threshold pressure value; and wherein the control unit is configured to activate the drive motor to reduce a size of the expandable cage implanted in the vessel when the central venous pressure falls below the threshold pressure value.

Clause 106. The method of any one of clauses 103-105, wherein the vessel is an azygos vein, the expandable cage configured to at least partially occlude flow from an azygos vein to a superior vena cava.

Clause 107. The method of any one of clauses 103-106, wherein the drive motor is implanted in a superior vena cava.

Clause 108. The method of any one of clauses 103-107, wherein collecting the one or more pressure measurements comprises measuring a first pressure measurement measured in an azygos vein using a first implantable sensor positioned at an outlet of a shunt and a second pressure measurement measured in a superior vena cava.

Clause 109. The method of any one of clauses 103-108, further comprising determining, by the control unit, a central venous pressure based on the one or more pressure measurements.

Clause 110. The method of any one of clauses 103-109, further comprising expanding the expandable cage when central venous pressure exceeds a threshold pressure value.

Clause 111. The method of any one of clauses 103-110, wherein the threshold pressure value is 15 mmHg.

Clause 112. The method of any one of clauses 103-111, further comprising collapsing the expandable cage when central venous pressure is below a threshold pressure value.

Clause 113. The method of clause 112, wherein the threshold pressure value is 5 mmHg.

Clause 114. The method of any one of clauses 103-111, further comprising activating a drive motor via the control unit to expand or collapse the expandable cage.

Clause 115. The method of any one of clauses 103-111, further comprising transmitting, by the control unit, the one or more pressure measurements to an external device.

Clause 116. The method of any one of clauses 103-111, further comprising charging, by a wireless charging coil, the control unit.

Clause 117. The method of any one of clauses 103-111, further comprising: collecting, by an accelerometer in the control unit implanted in a patient, one or more activity measurements; and activating, by the control unit, the expandable cage based at least in part on the one or more activity measurements.

Clause 118. The method of any one of clauses 103-111, further comprising: receiving, by an electrocardiogram in the control unit implanted in a patient, one or more electrical activity measurements; and activating, by the control unit, the expandable cage based at least in part on the one or more electrical activity measurements.

Clause 119. A method of implanting an occluder system, the method comprising: advancing an occluder system over a guidewire; positioning an expandable cage of the occluder system in an azygos vein to at least partially occlude blood flow toward a superior vena cava; anchoring the expandable cage using an anchoring element positioned in an azygos vein and distal of the expandable cage; positioning a drive line of the occluder system in a superior vena cava; and subcutaneously implanting a control unit, the drive line extending between the expandable cage and the control unit.

Clause 120. The method of clause 119, further comprising positioning the drive line of the occluder system at least partially in a right subclavian vein.

Clause 121. The method of any one of clauses 119-120, further comprising disconnecting the anchoring element from the expandable cage.

Clause 122. A system comprising one or more features of the foregoing description.

Clause 123. A method comprising one or more features of the foregoing description.

### ADDITIONAL CONSIDERATIONS AND TERMINOLOGY

Although certain methods have been described herein in connection with the azygos vein and the pulmonary artery, the systems described herein can also be used in other locations to redirect flow from an artery to a vein. Moreover, although the systems have been described herein as including an occluder system and a shunt, either of these components can be used independently. In an example, the occluder system can be effective in treating chronic refractory angina without a shunt. Chronic refractory angina can be caused by insufficient blood flow to the heart muscle. The occluder can be placed in the coronary sinus to occlude blood flow leaving the myocardium. This can cause an increase in blood pressure in the heart. The occluder can be adjusted in real-time. The occluder system can intervene and relieve potential angina symptoms before they occur or during occurrence.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the described embodiments, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

1-43. (canceled)

44. A system for treating heart failure of a patient, the system comprising:
a shunt configured to direct blood flow from a pulmonary artery to an azygos vein;
an expandable occluder configured to be positioned within the azygos vein to at least partially occlude blood flow through the azygos vein into a superior vena cava; and
an implantable control unit configured to regulate expansion of the expandable occluder to cause blood flow from the pulmonary artery through the shunt to the azygos vein in a retrograde direction through the azygos vein.

45. The system of claim 44, wherein the shunt comprises a check valve.

46. The system of claim 44, wherein the shunt is configured to passively allow blood to flow from the pulmonary artery to the azygos vein.

47. The system of claim 44, wherein the shunt comprises a first flared end configured to seal against a puncture in the pulmonary artery and a second flared end configured to seal against a puncture in the azygos vein.

48. The system of claim 44, further comprising an expandable stent configured to engage inner walls of the azygos vein.

49. The system of claim 44, further comprising a drive motor connectable to the expandable occluder, the drive motor configured to expand and collapse the expandable occluder.

50. The system of claim 44, wherein the control unit is configured to be implanted subcutaneously.

51. The system of claim 44, further comprising one or more implantable pressure sensors, the implantable control unit configured to receive pressure measurements from the one or more implantable pressure sensors, the implantable control unit configured to control the expandable occluder based on the pressure measurements.

52. The system of claim 44, wherein the implantable control unit comprises an accelerometer and/or an electrocardiogram.

53. The system of claim 44, wherein the expandable occluder comprises:
a first cap;
a second cap;
a plurality of members, each member comprising a first end positioned on the first cap and a second end positioned on the second cap; and
a drive line attached to the first cap, the drive line configured to:
rotate the first cap with respect to the second cap in a first direction to cause the plurality of members to expand radially outward, and
rotate the first cap with respect to the second cap in a second direction to cause the plurality of members to expand radially inward.

54. A method of treating heart failure of a patient, the method comprising:
positioning a shunt to direct blood flow from a pulmonary artery to an azygos vein;
positioning an expandable occluder within the azygos vein to at least partially occlude blood flow through the azygos vein into a superior vena cava; and
expanding the expandable occluder based on feedback received from one or more pressure sensors to at least partially occlude blood flow through the azygos vein such that blood flows from the pulmonary artery through the shunt to the azygos vein in a retrograde direction through the azygos vein.

55. The method of claim 54, wherein receiving the feedback from the one or more pressure sensors comprises measuring a first pressure measurement in the azygos vein using a first implantable sensor positioned in the azygos vein and a second pressure measurement in a superior vena cava.

56. The method of claim 54, wherein expanding the expandable occluder based on feedback received from the one or more pressure sensors comprises expanding the expandable occluder when central venous pressure exceeds a threshold pressure value.

57. The method of claim 56, wherein the threshold pressure value is 15 mmHg.

58. The method of claim 54, further comprising collapsing the expandable occluder when central venous pressure falls below a threshold pressure value.

59. The method of claim 58, wherein the threshold pressure value is 5 mmHg.

60. The method of claim 54, further comprising:
collecting, by an accelerometer, one or more activity measurements; and
expanding the expandable occluder based at least in part on the one or more activity measurements.

61. The method of claim 54, further comprising:
receiving, by an electrocardiogram, one or more electrical activity measurements; and
expanding the expandable occluder based at least in part on the one or more electrical activity measurements.

62. The method of claim 54, wherein expanding the expandable occluder comprises moving, by a drive motor, a first end of the expandable occluder closer to a second end of the expandable occluder and moving, by the drive motor, the first end of the expandable occluder away from the second end of the expandable occluder.

63. The method of claim 54, wherein expanding the expandable occluder comprises:
rotating, with a drive line attached to a first cap of the expandable occluder, the first cap with respect to a second cap of the expandable occluder.
